(19) United States
(12) Patent Application Publication
(10) Pub. No.: US 2026/0002211 A1
(43) Pub. Date: Jan. 1, 2026

(54) BIOMARKER COMPOSITION FOR DIAGNOSING STROKE OR POST-STROKE NEUROLOGICAL DAMAGE, FOR DIAGNOSING THE SEVERITY OF STROKE OR POST-STROKE NEUROLOGICAL DAMAGE, AND FOR PREDICTING STROKE PROGNOSIS

(71) Applicant: THE CATHOLIC UNIVERSITY OF KOREA INDUSTRY-ACADEMIC COOPERATION FOUNDATION, Seoul (KR)

(72) Inventors: Jae Sang OH, Gyeonggi-do (KR); Dong-Hun LEE, Incheon (KR); Eunchae LEE, Seoul (KR)

(21) Appl. No.: 18/945,687

(22) Filed: Nov. 13, 2024

(30) Foreign Application Priority Data
Jun. 26, 2024 (KR) 10-2024-0084045

(51) Int. Cl. C12Q 1/6883

(52) U.S. Cl. CPC C12Q 1/6883; C12Q 2600/112; C12Q 2600/158

(57) ABSTRACT

The present invention relates to a biomarker composition for diagnosing stroke or post-stroke neurological damage, for diagnosing the severity of stroke or post-stroke neurological damage, and for predicting stroke prognosis, and the like. Using an ischemic stroke mouse disease model, a tissue damage analysis and a behavioral analysis were performed, and the mRNA level expression was confirmed, and as a result, Prox1 or Dcx could be selected as a biomarker. In addition, it was confirmed that the expression of Prox1 or Dcx increased as the severity of stroke or post-stroke neurological damage was higher, and thus the present invention is expected to be useful for diagnosing stroke or post-stroke neurological damage, for diagnosing the severity of stroke or post-stroke neurological damage, and for predicting stroke prognosis.

### CROSS-REFERENCE TO RELATED APPLICATION

This application claims priority to and the benefit of Korean Patent Application No. 10-2024-0084045, filed on Jun. 26, 2024, the disclosure of which is incorporated herein by reference in its entirety.

### REFERENCE TO SEQUENCE LISTING SUBMITTED ELECTRONICALLY

The instant application contains a Sequence Listing which has been submitted electronically in XML format and is hereby incorporated by reference in its entirety. The Sequence Listing is written in the accompanying XML text file titled: “206132-0182-00US_SequenceListing.XML”; created on Oct. 25, 2024, and 21,504 bytes in size

### BACKGROUND

### 1. Field of the Invention

The present invention relates to a biomarker composition for diagnosing stroke or post-stroke neurological damage, for diagnosing the severity of stroke or post-stroke neurological damage, and for predicting stroke prognosis.

### 2. Discussion of Related Art

Stroke is a general term for a sudden, local neurological deficit caused by abnormal blood flow to the brain. According to the 2021 statistics of the Korean Statistical Information Service (KOSIS), the number of stroke patients in 2021 increased 6.6% on a yearly basis to 120,305 (as of Jan. 11, 2023), and according to the 2022 research report of the Korea Disease Control and Prevention Agency (KDCA), the treatment cost of cerebral infarction in 2019 was 2,009,200,000 KRW, an increase of about 1.54 times from 1,361,000,000 KRW in 2015. In addition, the number of people who received emergency room treatment for stroke in Korea in 2019 was 120,584, an increase of about 29.7% from 93,670 in 2014, and the proportion of the population who received treatment for stroke increased from 1.7% in 2014 to 1.9% in 2019. In the United States, stroke is the third leading cause of death and the leading cause of disability. About 20% of stroke patients require long-term hospitalization for more than three months, and 15% to 30% of them have permanent disability. According to the 2022 report published by the World Stroke Organization (WSO), the cost of stroke treatment worldwide in 2017 was 45.1 billion dollars.

In addition, the 2022 report published by the WSO pointed out high systolic blood pressure as the single biggest risk factor for stroke worldwide. In addition, cerebral small vessel disease (CSVD) may cause cerebral ischemic problems, beginning with microinfarcts such as lacunar infarcts. Recently, research is also being conducted on the effects of air pollution on stroke.

Meanwhile, some stroke patients continue to suffer from sequelae even after receiving treatment. The Korean Stroke Society reported in a survey conducted with stroke patients that 98% of the patients returned to the hospital with hemiplegia, speech disorders, visual impairment, dizziness, severe headaches, and the like. Among the 318,127 acute stroke hospitalizations that occurred in the United States from October 2014 to March 2018, 15.7% received recombinant tissue plasminogen activator (rTPA) and 5.4% received mechanical thrombectomy. Nevertheless, more than half of patients receiving acute treatment progressed to long-term disability. Post-stroke cognitive impairment and dementia are still prevalent, and 70% of stroke survivors suffer from cognitive impairment as one of the sequelae. However, biomarkers related to neurological damage among the sequelae have not been well studied. Therefore, research on this is required.

### RELATED ART DOCUMENTS

### Patent Document

(Korea Patent Publication) No. 10-2020-0034619

### SUMMARY OF THE INVENTION

The present invention is directed to providing a method of diagnosing stroke or post-stroke neurological damage, including:
(a) measuring the expression level of one or more mRNAs selected from the group consisting of Prox1 and Dcx from a biological sample isolated from a subject;
(b) comparing the expression level of the mRNAs with that of a control biological sample;
(c) determining that the subject has a stroke or post-stroke neurological damage when the mRNA level of Prox1 is at least 2.03±0.64 as measured in Step (b); or
determining that the subject has a stroke or post-stroke neurological damage when the mRNA level of DCX is at least 1.63±0.58 as measured in Step (b); and
(d) treating the subject determined to have a stroke or post-stroke neurological damage in Step (c) by administering a pharmaceutically effective amount of a preparation for treating the subject.

Also, the present invention is directed to providing a method of treating stroke or post-stroke neurological damage, including:
(a) measuring the expression level of one or more mRNAs selected from the group consisting of Prox1 and Dcx from a biological sample isolated from a subject;
(b) comparing the expression level of the mRNAs with that of a control biological sample;
(c) determining that the severity of stroke or post-stroke neurological damage is high when the mRNA level of Prox1 is at least 2.03±0.64 as measured in Step (b); or
determining that the severity of stroke or post-stroke neurological damage is high when the mRNA level of DCX is at least 1.63±0.58 as measured in Step (b); and
(d) treating the subject determined to have high severity of stroke or post-stroke neurological damage in Step (c) by administering a pharmaceutically effective amount of a preparation for treating the subject.

Also, the present invention is directed to providing a method of treating stroke or post-stroke neurological damage, including:
(a) measuring the expression level of one or more mRNA selected from the group consisting of Prox1 and Dcx or a gene encoding the protein from a biological sample isolated from an individual;
(b) comparing the expression level of the mRNA with that of a control biological sample;
(c) determining that the subject has a stroke or post-stroke neurological damage when the mRNA level of Prox1 is at least 2.03±0.64 as measured in Step (b); or
determining that the subject has a stroke or post-stroke neurological damage when the mRNA level of DCX is at least 1.63±0.58 as measured in Step (b); and
(d) treating the subject determined to have a stroke or post-stroke neurological damage in Step (c) by administering a pharmaceutically effective amount of a preparation for treating the subject.

However, the technical tasks to be achieved by the present invention are not limited to the above-described tasks, and other tasks not mentioned may be clearly understood by one of ordinary skill in the art to which the present invention pertains from the description below.

The present invention provides a method of diagnosing stroke or post-stroke neurological damage, including:
(a) measuring the expression level of one or more mRNAs selected from the group consisting of Prox1 and Dcx from a biological sample isolated from a subject;
(b) comparing the expression level of the mRNAs with that of a control biological sample;
(c) determining that the subject has a stroke or post-stroke neurological damage when the mRNA level of Prox1 is at least 2.03±0.64 as measured in Step (b); or
determining that the subject has a stroke or post-stroke neurological damage when the mRNA level of DCX is at least 1.63±0.58 as measured in Step (b); and
(d) treating the subject determined to have a stroke or post-stroke neurological damage in Step (c) by administering a pharmaceutically effective amount of a preparation for treating the subject.

In one embodiment of the present invention, the stroke may be one or more selected from the group consisting of hemorrhagic stroke and ischemic stroke, but is not limited thereto.

In another embodiment of the present invention, the stroke or post-stroke neurological damage may be characterized by any one or more selected from the group consisting of:
(a) occurrence of a symptom of decreased blood flow reperfusion;
(b) occurrence of a symptom of increased cerebral infarction area;
(c) occurrence of a general motor impairment symptom;
(d) occurrence of a sensorimotor impairment symptom;
(e) occurrence of long-term cognitive memory impairment; and
(f) occurrence of death or arrangement atrophy of hippocampal cells, but is not limited thereto.

In another embodiment of the present invention, the stroke or post-stroke neurological damage may be characterized by an increase in the mRNA level of any one or more selected from the group consisting of:
vascular cell adhesion molecule 1 (Vcam1), intercellular adhesion molecule 1 (Icam1), interleukin-1 beta (IL-1β), monocyte chemoattractant protein 1 (Mcp1), and C-C chemokine receptor 2 (Ccr2), but is not limited thereto.

In another embodiment of the present invention, the stroke or post-stroke neurological damage may be due to cerebral artery occlusion, but is not limited thereto.

In another embodiment of the present invention, the cerebral artery may be any one or more selected from the group consisting of anterior cerebral artery, middle cerebral artery, and posterior cerebral artery, but is not limited thereto.

Furthermore, provided is a method of treating stroke or post-stroke neurological damage, including:
(a) measuring the expression level of one or more mRNAs selected from the group consisting of Prox1 and Dcx from a biological sample isolated from a subject;
(b) comparing the expression level of the mRNAs with that of a control biological sample;
(c) determining that the severity of stroke or post-stroke neurological damage is high when the mRNA level of Prox1 is at least 2.03±0.64 as measured in Step (b); or
determining that the severity of stroke or post-stroke neurological damage is high when the mRNA level of DCX is at least 1.63±0.58 as measured in Step (b); and
(d) treating the subject determined to have high severity of stroke or post-stroke neurological damage in Step (c) by administering a pharmaceutically effective amount of a preparation for treating the subject.

Furthermore, provided is a method of treating stroke or post-stroke neurological damage, including:
(a) measuring the expression level of one or more mRNA selected from the group consisting of Prox1 and Dcx or a gene encoding the protein from a biological sample isolated from an individual;
(b) comparing the expression level of the mRNA with that of a control biological sample;
(c) determining that the subject has a stroke or post-stroke neurological damage when the mRNA level of Prox1 is at least 2.03±0.64 as measured in Step (b); or
determining that the subject has a stroke or post-stroke neurological damage when the mRNA level of DCX is at least 1.63±0.58 as measured in Step (b); and
(d) treating the subject determined to have a stroke or post-stroke neurological damage in Step (c) by administering a pharmaceutically effective amount of a preparation for treating the subject.

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings, in which:

FIGS. 1A to 1C illustrate the outline of the MCAO mouse model experimental design (MCAO: middle cerebral artery occlusion; NS: neurological score; OFT: open field test; ART: adhesive removal test; RT-PCR: real-time polymerase chain reaction; TTC: triphenyltetrazolium chloride; H&E: hematoxylin and eosin; ACA: anterior cerebral artery; MCA: middle cerebral artery; PCA: posterior cerebral artery; BA: basilar artery; ICA: internal carotid artery; PPA: pterygopalatine artery; ECA: external carotid artery; CCA: common carotid artery; BPU: blood perfusion units);

FIG. 1A is a diagram showing that the neurological score was measured in all MCAO mice after surgery, behavioral experiments were conducted sequentially from low-stress experiments to high-stress experiments from day 7, and immediately after the behavioral experiments were completed, euthanasia was performed to extract brain tissues, the mRNA level expression was confirmed, and sampling was performed for histological staining;

FIG. 1B illustrates the MCAO surgery for inducing ischemic stroke in mice. Specifically, the blood flow in the CCA, ICA, and ECA was blocked using vascular clamps, and then an MCAO suture was inserted through the incision site of the ECA to achieve M1 region occlusion;

FIG. 1C illustrates the MCAO procedure and blood flow measurement. Specifically, the CCA and ICA were blocked using vascular clamps, and the upper part of the ECA was permanently ligated with a suture to block all blood flow. The ECA was incised with microscissors, the MCAO suture was inserted, and the ICA clamp was removed. Then, a filament was advanced, and when the filament reached the M1 region of the MCA, occlusion was achieved, and it was maintained for 30 minutes. After 30 minutes, the filament was withdrawn to achieve blood reperfusion. The blood flow of the MCA was 400 BPU on average just before MCAO (−1 on the x-axis of the graph), and 0 BPU during the occlusion process. After the reperfusion, the blood flow was 300 to 400 BPU depending on the individual, indicating that reperfusion was achieved;

FIGS. 2A to 2D show the results of measuring cerebral infarction area, neurological score, and neuroinflammation (*p<0.05, **p<0.01, ***p<0.001, ****p<0.0001. WT: wild type, MCAO: middle cerebral artery occlusion, NS: neurological score, OFT: open field test, ART: adhesive removal test, Vcam1: vascular cell adhesion molecule 1, Icam1: intercellular adhesion molecule 1, IL: interleukin, Mcp1: monocyte chemoattractant protein 1, ccr2: c-c chemokine receptor 2, TTC: triphenyltetrazolium chloride);

FIG. 2A shows the results of measuring the cerebral infarction area and NS in the MCAO group. The figure on the left shows the TTC staining results of the WT group and the MCAO1 and MCAO2 groups, and the figure on the right shows the results of measuring the infarction area in the MCAO1 and MCAO2 groups;

FIG. 2B is a diagram showing the results of NS measurements after MCAO treatment (WT: wild type, MCAO: middle cerebral artery occlusion);

FIG. 2C is a diagram showing the neuroinflammation-related mRNA levels of the MCAO group;

FIG. 2D shows the general motor and sensorimotor changes of the MCAO group. Specifically, the diagram shows the results of measuring the total distance traveled in the OFT and the adhesive removal latency of ART for the general motor measurement of the WT group and the MCAO group;

FIG. 3 is a diagram showing the results of measuring positive long-term cognitive memory in the MCAO group (***p<0.001. WT: wild type, MCAO: middle cerebral artery occlusion);

FIGS. 4A and 4B are diagrams showing histological changes in hippocampus and neural biomarker mRNA levels in the MCAO group (***p<0.001, ****p<0.0001. WT: wild type, MCAO: middle cerebral artery occlusion, Prox1: prospero homeobox 1, dcx: doublecortin);

FIG. 4A shows the results of hematoxylin and eosin (H&E) staining of the two groups, WT and MCAO, and a microscopic examination (x350) showing cell arrangement and apoptosis in the hippocampus region by H&E staining;

FIG. 4B shows the mRNA levels of Prox1 and Dcx in the WT and MCAO groups; and

FIG. 5 schematically illustrates an experiment to confirm the behavioral, histological, and molecular biological changes due to neurological damage after ischemic stroke. A mouse model mimicking human ischemic stroke was produced by inducing MCAO through surgical intervention, and the mice were assessed for the NS and classified into low- or high-symptom groups according to the severity of the symptoms. The groups were then assessed for the effects of ischemic stroke through behavioral and histological analyses (WT: wild type; MCAO: middle cerebral artery occlusion; NS: neurological score; Prox1: prospero homeobox 1; Dcx: doublecortin; PCR: polymerase chain reaction; TTC: triphenyltetrazolium chloride; H&E: hematoxylin and eosin).

### DETAILED DESCRIPTION OF EXEMPLARY EMBODIMENTS

The present invention provides a method of diagnosing stroke or post-stroke neurological damage, including:
(a) measuring the expression level of one or more mRNAs selected from the group consisting of Prox1 and Dcx from a biological sample isolated from a subject;
(b) comparing the expression level of the mRNAs with that of a control biological sample;
(c) determining that the subject has a stroke or post-stroke neurological damage when the mRNA level of Prox1 is at least 2.03±0.64 as measured in Step (b); or
determining that the subject has a stroke or post-stroke neurological damage when the mRNA level of DCX is at least 1.63±0.58 as measured in Step (b); and
(d) treating the subject determined to have a stroke or post-stroke neurological damage in Step (c) by administering a pharmaceutically effective amount of a preparation for treating the subject.

In one embodiment of the present invention, the stroke may be one or more selected from the group consisting of hemorrhagic stroke and ischemic stroke, but is not limited thereto.

In another embodiment of the present invention, the stroke or post-stroke neurological damage may be characterized by any one or more selected from the group consisting of:
(a) occurrence of a symptom of decreased blood flow reperfusion;
(b) occurrence of a symptom of increased cerebral infarction area;
(c) occurrence of a general motor impairment symptom;
(d) occurrence of a sensorimotor impairment symptom;
(e) occurrence of long-term cognitive memory impairment; and
(f) occurrence of death or arrangement atrophy of hippocampal cells, but is not limited thereto.

In another embodiment of the present invention, the stroke or post-stroke neurological damage may be characterized by an increase in the mRNA level of any one or more selected from the group consisting of:
vascular cell adhesion molecule 1 (Vcam1), intercellular adhesion molecule 1 (Icam1), interleukin-1 beta (IL-1β), monocyte chemoattractant protein 1 (Mcp1), and C-C chemokine receptor 2 (Ccr2), but is not limited thereto.

In another embodiment of the present invention, the stroke or post-stroke neurological damage may be due to cerebral artery occlusion, but is not limited thereto.

In another embodiment of the present invention, the cerebral artery may be any one or more selected from the group consisting of anterior cerebral artery, middle cerebral artery, and posterior cerebral artery, but is not limited thereto.

In the present invention, the level of Prox1 may be used interchangeably with the expression level of one or more selected from the group consisting of Prox1 and a gene encoding Prox1, but is not limited thereto.

In the present invention, the level of Dcx may be used interchangeably with the expression level of one or more selected from the group consisting of Dcx and a gene encoding Dcx, but is not limited thereto.

In the present invention, Prox1 may be conserved throughout vertebrates and may play an essential role in the development process, but is not limited thereto. In addition, in the present invention, Prox1 may refer to a protein encoded by a Prox1 gene, and may be used as a term referring to both the gene form or the protein form according to the situation.

In one embodiment of the present invention, the Prox1 protein may be an amino acid sequence represented by SEQ ID NO: 1, but is not limited thereto. In addition, in one embodiment of the present invention, specific information regarding Prox1 may be found at the National Center for Biotechnology Information (NCBI) (NCBI Reference Sequence: NP_001347756.1).

In one embodiment of the present invention, the gene encoding Prox1 may be a base sequence represented by SEQ ID NO: 2, but is not limited thereto. In addition, in one embodiment of the present invention, specific information regarding Prox1 may be found at the NCBI (NCBI Reference Sequence: NM_001360827.2).

In the present invention, Dex may be a cytoplasmic protein including two doublecortin domains that bind to microtubules, and may play a role in directing the movement of neurons by regulating the organization and stability of microtubules, but is not limited thereto. In addition, in the present invention, Dex may refer to a protein encoded by the Dcx gene, and may be used as a term referring to both the gene form or protein form according to the situation.

In one embodiment of the present invention, the Dex protein may be an amino acid sequence represented by SEQ ID NO: 3, but is not limited thereto. In addition, in one embodiment of the present invention, specific information regarding Dcx may be found at the NCBI (NCBI Reference Sequence: NP_001103694.1).

In one embodiment of the present invention, the gene encoding Dcx may be a base sequence represented by SEQ ID NO: 4, but is not limited thereto. In addition, in one embodiment of the present invention, specific information regarding Dcx may be found at the NCBI (NCBI Reference Sequence: NM_001110222.1).

The sequences of SEQ ID Nos: 1 to 4 of the present invention are as shown in Table 1 below.

TABLE 1

| SEQ ID | | | |
|---|---|---|---|
| NO. | Name | Sequence | Species |
| 1 | Prox 1 | mpdhdstallsrqtkrrrvdigvkrtvgtasaffakaratffsamnpqgseqdveysvv | Mus |
| | protein | qhadgeksnvlrkllkransyedammpfpgatiisqllknnmnknggtepsfqasgls | musculus |
| | | stgsevhqedicsnssrdsppeclspfgrptmsqfdvdrlcdehlrakrarveniirgms | |
| | | hspsvalrgneneremapqsvspresyrenkrkqklpqqqqqsfqqlvsarkeqkree | |
| | | rrqlkqqledmqkqlrqlqekfyqvydstdsendedgdlsedsmrseildaraqdsvg | |
| | | rsdnemceldpgqfidraralireqemaenkpkregsnkerdhgpnslqpegkhlaet | |
| | | lkqelntamsqvvdtvvkvfsakpsrqvpqvfpplqipqarfavngenhnfhtanqrl | |
| | | qcfgdviipnpldtfgsvqmpsstdqtealplvvrknsseqsasgpatgghhqplhqsp | |
| | | lsatagfttpsfrhpfplplmaypfqsplgapsgsfsgkdraspesldltrdttslrtkmssh | |
| | | hlshhpcspahppstaeglslsliksecgdlqdmsdispysgsamqeglspnhlkkakl | |
| | | mffytrypssnmlktyfsdvkfnrcitsqlikwfsnfrefyyiqmekyarqaindgvtst | |
| | | eelsitrdcelyralnmhynkandfevperflevaqitlreffnaiiagkdvdpswkkai | |
| | | ykvickldsevpeifkspnclqellhe | |
| 2 | Gene | atgcctgaccatgacagcacagccctcttaagccggcaaaccaagaggagaagggttga | Mus |
| | encoding | cattggagtgaaaaggacggtagggacagcatctgcattttttgctaaggcaagggcaac | musculus |
| | Prox 1 | atttttcagtgccatgaatccccaaggttcagagcaggatgttgaatattctgtggtgcaaca | |
| | | cgcagatggggaaaagtcgaacgtactccgcaagctgctgaagagggcgaactcgtatg | |
| | | aagatgccatgatgccttttccaggagcaactataatttcccagctgttgaaaaataacatga | |
| | | acaaaaacggtggcaccgagcccagtttccaagccagcggactctctagcacaggctcc | |
| | | gaagtacatcaggaggatatatgtagcaactcttcaagagacagccccccagagtgtcttt | |
| | | ccccttttggcaggcctactatgagccagtttgatgtggatcgcttatgtgatgagcacctga | |
| | | gagcaaagcgcgcccgggttgagaatatcattcggggtatgagccattcccccagtgtgg | |
| | | cattaaggggcaatgaaaacgaaagagagatggccccgcagtctgtgagtccccgagaa | |
| | | agttacagagaaaacaaacgcaagcagaagctgccccagcagcagcaacagagtttcc | |
| | | agcagctggtttcagcccgaaaagaacagaagcgagaggagcgccgacagctgaaac | |
| | | agcagctggaagacatgcagaagcagctgcgccagctgcaggagaagttctaccaggt | |
| | | ctatgacagcacagactccgaaaatgatgaagatggcgacctgtctgaagacagcatgcg | |
| | | ctcggagatcctggatgcacgggcccaggactcggtggggcgctcagacaatgagatgt | |
| | | gtgagctggacccagggcagttcatcgacagggcccgagccctaatcagggagcagga | |
| | | gatggctgagaacaagcctaagcgagaaggcagcaacaaagaaagagaccacgggcc | |
| | | aaactccttgcagccagaaggcaagcatctggcagagaccttaaaacaggagctgaaca | |
| | | cggccatgtcgcaggttgtggacacggtggtcaaagtcttctcagccaaaccctctcgcca | |
| | | ggttcctcaggtcttcccacctctccagatcccccaggccagattcgcagtcaacggggaa | |
| | | aaccacaatttccacacggccaaccagcgcctgcaatgctttggtgatgtcatcattccgaa | |
| | | ccccttggacacctttggcagtgtgcagatgcctagttccacagaccagacggaagccctt | |
| | | cccctggtggtccgaaaaaactcatccgagcaatctgcctctggcccggccactggcggc | |
| | | caccaccagcccctgcaccagtcacccctctccgccactgcaggcttcaccacccctagc | |
| | | ttccgccatccctttcccctgcccttgatggcttatccatttcagagtccactaggtgctccct | |
| | | ccggctccttctcggggaaggacagagcctctcctgagtccttagacttgactcgggacac | |
| | | aacaagtctgaggaccaagatgtcatcacaccatctgagccaccacccctgttcaccagc | |
| | | acacccacccagcaccgcagaaggactctctttgtcactcataaagtctgagtgtggagat | |
| | | cttcaagatatgtccgacatctcaccttattcaggaagcgcaatgcaggaagggctatcac | |
| | | ccaatcacttgaaaaaggcaaaactcatgttcttttacacccgctaccccagctccaacatg | |
| | | ctgaagacctacttctcggacgtgaagttcaacagatgcattacctcgcagctcatcaagtg | |
| | | gttcagcaatttccgtgagttttactatatccagatggagaagtatgcgcgtcaagccatcaa | |
| | | tgatggagtcaccagtacagaagagctctccatcaccagggattgtgagctataccgagc | |
| | | cctcaacatgcactacaacaaagcaaatgactttgaggttccagagagattcctggaagtt | |
| | | gcgcagatcacgttacgggagtttttcaatgccatcatcgcgggcaaagatgttgatccttc | |
| | | ctggaagaaggccatttacaaggtcatctgcaagctggatagtgaagttcctgagattttca | |
| | | aatcccctaactgcctacaagaactccttcacgagtag | |
| 3 | Dcx | meldfghfderdkasrnmrgsrmnglpspthsahcsfyrtrtlqalsnekkakkvrfyr | Mus |
| | protein | ngdryfkgivyavssdrfrsfdalladltrslsdninlpqgvryiytidgsrkigsmdelee | musculus |
| | | gesyvcssdnffkkveytknvnpnwsvnvktsanmkapqslassnsaqarenkdfv | |
| | | rpklvtiirsgvkprkavrvllnkktahsfeqvltditeaikletgvvkklytldgkqvtclh | |
| | | dffgdddvfiacgpekfryaqddfsldenecrvmkgnpsaaagpkasptpqktsaks | |
| | | pgpmrrskspadsgndqdangtsssqlstpkskqspistptspgslrkhkdlylplsldd | |
| | | sdslgdsm | |
| 4 | Gene | atggaacttgattttggacattttgacgaacgagacaaagcatctagaaatatgagagggtc | Mus |
| | encoding | acggatgaatggacttccaagtcccactcatagtgcccactgtagcttctacagaaccaga | musculus |
| | Dcx | accttgcaggcattaagtaatgagaagaaggccaagaaggtacgtttctaccgcaatggg | |
| | | gaccgttacttcaaggggattgtgtacgctgtttcttctgaccgttttcgtagttttgatgcgtt | |
| | | gctggctgacctgacccgatccttgtctgacaacattaacctgcctcagggagtgcgctacat | |
| | | ttataccattgacggatccaggaagattggaagcatggatgaactggaagaaggggaaa | |
| | | gctatgtctgctcctcagacaacttctttaaaaaggttgagtacaccaagaatgtcaacccca | |
| | | actggtctgtcaacgtaaagacatctgccaacatgaaagccccccagtccttggctagcag | |
| | | caacagtgctcaagccagagagaacaaggactttgtgcgccccaaacttgtgaccatcatt | |
| | | cgcagcggggtgaagccacggaaggctgtgcgcgtgcttctcaacaagaaaacagccc | |
| | | actctttcgagcaggtcctgactgacatcacagaagcgatcaaactggaaaccggagttgt | |
| | | caaaaaactctacacccttgatggaaagcaggtcacctgtctccatgatttctttggtgatga | |
| | | tgatgtgttcattgcttgtggtcctgaaaaattccgctatgctcaagatgatttctccttggatg | |
| | | agaatgaatgcagagtcatgaaagggaatccatctgccgcagctggcccaaaggcttccc | |
| | | caacacctcaaaagacatctgctaaaagcccaggcccaatgcgccgcagcaagtctcca | |
| | | gctgactcaggtaacgaccaagacgcaaatggaacttccagcagtcagctctcaacacct | |
| | | aagtcaaagcagtctcctatctctacacccacaagccctggaagtctgcggaagcacaag | |
| | | gtagacctgtacctgccgctgtcattggatgactctgattcacttggcgattccatgtga | |

In the present invention, the Prox1 protein and the Dex protein may include an amino acid sequence having a sequence homology of 70% or more, preferably 80% or more, more preferably 90% or more, and most preferably 95% or more with one or more of the amino acid sequences represented by SEQ ID NO: 1 and SEQ ID NO: 3, respectively. In other words, the Prox1 protein and the Dcx protein may include an amino acid sequence having a sequence homology of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% with one or more of the amino acid sequences represented by SEQ ID NO: 1 and SEQ ID NO: 3, respectively. These are only representative examples of the Prox1 protein and the Dcx protein, and the types of Prox1 protein and Dex protein are not limited by the above sequences.

In the present invention, the gene encoding Prox1 and the gene encoding Dcx may be encoded by a base sequence having a sequence homology of 70% or more, preferably 80% or more, more preferably 90% or more, and most preferably 95% or more with the base sequences represented by SEQ ID NO: 2 and SEQ ID NO: 4, respectively. In other words, the gene encoding Prox1 and the gene encoding Dcx may be encoded by a base sequence having a sequence homology of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% with the base sequences represented by SEQ ID NO: 2 and SEQ ID NO: 4, respectively. These are only representative examples of the gene encoding Prox1 and the gene encoding Dcx, and the types of the gene encoding Prox1 and the gene encoding Dcx are not limited by the above sequences.

In the present invention, stroke may be a general term for local neurological deficit symptoms suddenly induced due to abnormal blood flow to the brain, and may be used interchangeably with cerebrovascular accident (CVA), but is not limited thereto. In addition, in the present invention, stroke risk factors may be immutable factors such as age and family history, and general vascular risk factors such as hypertension, obesity, coronary artery stenosis, diabetes, smoking, myocardial infarction, excessive alcohol consumption, and bacterial infection, but are not limited thereto.

In the present invention, abnormal blood flow to the brain may cause problems in the supply of glucose, which is the brain's energy source, and when there is a problem in the supply of glucose, which is the brain's only energy source, brain tissue may easily undergo necrosis, but is not limited thereto. In addition, brain tissue damage due to abnormal cerebral blood flow may cause symptoms of brain cognitive impairment, but is not limited thereto.

In the present invention, some patients who have suffered a stroke may have disabilities remaining even after treatment, more than half of patients receiving acute treatment may progress to long-term disabilities, and post-stroke cognitive impairment and dementia are still prevalent, and thus 70% of stroke survivors may experience cognitive impairment as one of the sequelae.

In the present invention, symptoms of stroke may vary depending on the affected area. Specifically, when the central nervous system is affected, facial hemiplegia and numbness, excessive reflexes due to decreased or increased muscle tension, problems with smell, taste, hearing, vision, and the like may occur, but the symptoms are not limited thereto. In addition, when the cerebral cortex is affected, aphasia, visual field defects, memory deficits, and disorganized thinking involving the central nervous system pathway may occur, and when the cerebellum is affected, symptoms of dizziness or imbalance accompanied by abnormal gait may occur, but the symptoms are not limited thereto.

In the present invention, stroke may be classified into two types, which are hemorrhagic stroke and ischemic stroke, but is not limited thereto.

In the present invention, hemorrhagic stroke includes intracerebral hemorrhage, intradural hemorrhage, and intraventricular hemorrhage that occur within the brain itself, and subarachnoid hemorrhage that occurs under the arachnoid membrane outside the brain tissue, and hemorrhagic stroke may occur in the background of cerebral amyloid angiopathy, cerebral aneurysm, and the like, but is not limited thereto.

In the present invention, ischemic stroke refers to a decrease in blood supply to a local area of the brain and the occurrence of functional impairment in that region. The decrease in blood supply due to ischemic stroke may be caused by thrombosis due to a thrombus generated in a local region, obstruction due to an embolism that has moved from another region, and the like, but is not limited thereto. In addition, in the present invention, ischemic stroke may be used interchangeably with cerebral infarction, but is not limited thereto.

In the present invention, the central nervous system affected by ischemic stroke may undergo aphasia, visual defects, memory deficits, or thinking disorders, but is not limited thereto. Specifically, decreased blood supply to the brain due to vascular damage caused by ischemic stroke may impair the supply process of glucose, which is the brain's energy source, and cause necrosis of brain tissue, whose the only energy source is glucose, and damage to brain tissue due to abnormal cerebral blood flow may cause cognitive impairment symptoms, but is not limited thereto. More specifically, brain tissue affected by ischemia may rely on anaerobic metabolism to replenish energy, and the anaerobic metabolism releases lactic acid as a byproduct, which may disrupt the acid-base balance in brain tissue and potentially destroy cells. Furthermore, the lack of adenosine triphosphate (ATP) may cause the breakdown of energy-dependent processes such as ion pumps, resulting in indiscriminate extracellular release of the excitatory neurotransmitter glutamate, and excessive glutamate acts on neuronal receptors such as N-methyl-D-aspartate (NMDA) receptors to cause excessive calcium influx, and excessive calcium may induce mitochondrial death, which may lead to further lack of energy. In addition, ischemic conditions may induce the production of oxygen free radicals and reactive oxygen species (ROS), which may damage cells, but are not limited to thereto.

In the present invention, ischemic stroke may not necessarily occur due to an increase in inflammatory factors, but is not limited thereto.

In the present invention, the method of treating ischemic stroke includes the use of a thrombolytic agent and a method of achieving cerebral reperfusion through mechanical thrombectomy, but is not limited thereto. Specifically, the thrombolytic agent may be a drug that may achieve reperfusion of blood flow by dissolving a thrombus that blocks a blood vessel.

In the present invention, the ischemic stroke cascade may be caused by ischemic stroke, and the ischemic cascade may induce the production of reactive oxygen and ROS that may cause not only ischemic damage but also cell damage, but is not limited thereto. In one embodiment of the present invention, a neurological damage biomarker that may prove neurological (system) damage that occurs along with the progression of the ischemic cascade may be disclosed, but is not limited thereto.

In the present invention, post-stroke neurological damage may be associated with small vessel diseases (SVD) as described in Table 2 below, but is not limited thereto. In addition, each of these diseases is caused by intravascular deposition or inflammatory damage and may develop into stroke, but is not limited thereto. In addition, in the present invention, the neurological damage may be used interchangeably with nerve damage, cranial nerve damage, and hippocampal damage, but is not limited thereto.

TABLE 2

| Disease | Neuropathological characteristics |
|---|---|
| Arteriosclerosis | Vascular lumen stenosis |
| Cerebral amyloid | Amyloid-beta deposition in blood vessel walls |
| angiopathy | |
| White matter | White matter thinning, myelin loss, axonal damage, |
| damage | oligodendrocyte loss, reactive astrocytes, |
| | macrophages, perivascular space dilatation, blood- |
| | brain barrier leakage, neuroinflammation |
| Lacunar infarct | Neuroinflammation of stellate cells |
| Enlarged | Dilated perivascular space filled with |
| perivascular space | extracellular matrix |

In the present invention, the biomarker related to post-stroke neurological damage may be a biomarker that diagnoses the severity of stroke or post-stroke neurological damage by differences in molecular biological expression, and the biomarker of the present invention may be used to confirm cognitive dysfunction caused by a specific factor, but is not limited thereto.

In the present invention, the severity of stroke or post-stroke neurological damage may refer to the severity of behavioral disorder, the severity of (long-term) cognitive dysfunction, the severity of nerve cell tissue damage, the severity of brain tissue damage, the severity of brain nervous system damage, or the severity of spinal nervous system damage, but is not limited thereto. In addition, in the present invention, the severity of stroke or post-stroke neurological damage may be proportional to the increase in the mRNA expression levels of Prox1 and Dcx, but is not limited thereto.

In the present invention, diagnosis refers to confirming the presence or characteristics of a pathological condition. According to the purpose of the present invention, diagnosis includes confirming the presence, occurrence, or possibility of occurrence (risk of occurrence) of stroke or post-stroke neurological damage, but is not limited thereto, and also includes confirming the severity of stroke or post-stroke neurological damage.

More specifically, the term “diagnosis” as used herein includes determining the susceptibility of a subject to a particular disease or condition, determining whether a subject currently has a particular disease or condition, determining the prognosis of a subject with a particular disease or condition, or therametrics (e.g., monitoring the condition of a subject to provide information about treatment efficacy).

In the present invention, the severity may refer to the degree of increase or decrease in the risk of developing stroke or post-stroke neurological damage, the degree of progression of a lesion, and the degree of increase or decrease in the risk of recurrence. The degree of increase or decrease in the risk of developing includes the degree of risk of developing or the possibility of developing stroke or post-stroke neurological damage. The degree of progression of a lesion includes the degree of developing stroke or post-stroke neurological damage and progression of the lesion, resulting in mild to severe stroke or post-stroke neurological damage. In addition, the degree of increase or decrease in the risk of recurrence includes the degree of risk of recurrence or the possibility of recurrence after stroke or post-stroke neurological damage is determined to be completely cured. The severity of the stroke or post-stroke neurological damage may be expressed qualitatively and/or quantitatively, and the severity may be classified according to the degree.

In the present invention, the method of diagnosing the severity of stroke or post-stroke neurological damage may be used in place of a severity diagnosis method known in the art, or may be used in combination with a severity diagnosis method known in the art.

In the present invention, the biomarker refers to a marker capable of distinguishing a normal or pathological state or predicting and objectively measuring a therapeutic response. It was confirmed that the level of Prox1 or Dcx in biological samples of subjects with a stroke or post-stroke neurological damage according to the present invention was different in terms of the increase or decrease of each level compared to a normal control group or a stroke or post-stroke neurological damage experimental group, and thus it was proven that Prox1 or Dcx may be used as a biomarker not only for diagnosing stroke or post-stroke neurological damage or for diagnosing the severity but also for predicting stroke prognosis.

In the present invention, predicting prognosis may refer to predicting the degree of progression of stroke or post-stroke neurological damage. It may refer to predicting the probability of progression, deterioration, recurrence, maintenance, and the like of the pathological condition of stroke or post-stroke neurological damage through an increase or decrease in the level of the biomarker of the present invention.

In the present invention, blood flow reperfusion may refer to restoration of blood flow to an organ or tissue after blood flow has been blocked, but is not limited thereto. In addition, in one embodiment of the present invention, the blood flow reperfusion of a subject with a stroke may be 200 to 400, 200 to 390, 200 to 380, 200 to 370, 200 to 360, 200 to 350, 200 to 340, 200 to 330, 200 to 320, 200 to 310, 200 to 300, 250 to 400, 250 to 390, 250 to 380, 250 to 370, 250 to 360, 250 to 350, 250 to 340, 250 to 330, 250 to 320, 250 to 310, 250 to 300, 300 to 400, 300 to 390, 300 to 380, 300 to 370, 300 to 360, 300 to 350, 300 to 340, 300 to 330, 300 to 320, 300 to 310, 350 to 400, 350 to 390, 350 to 380, 350 to 370, and 350 to 360 blood perfusion units (BPU), but is not limited thereto.

In one embodiment of the present invention, the cerebral infarction area may be 10% to 60%, 10% to 58%, 10% to 56%, 10% to 54%, 10% to 52%, 10% to 50%, 10% to 48%, 10% to 46%, 10% to 44%, 10% to 42%, 10% to 40%, 10% to 38%, 10% to 36%, 10% to 34%, 10% to 32%, 10% to 30%, 10% to 28%, 10% to 26%, 10% to 24%, 10% to 22%, 10% to 20%, 15% to 60%, 15% to 58%, 15% to 56%, 15% to 54%, 15% to 52%, 15% to 50%, 15% to 48%, 15% to 46%, 15% to 44%, 15% to 42%, 15% to 40%, 15% to 38%, 15% to 36%, 15% to 34%, 15% to 32%, 15% to 30%, 15% to 28%, 15% to 26%, 15% to 24%, 15% to 22%, 15% to 20%, 20% to 60%, 20% to 58%, 20% to 56%, 20% to 54%, 20% to 52%, 20% to 50%, 20% to 48%, 20% to 46%, 20% to 44%, 20% to 42%, 20% to 40%, 20% to 38%, 20% to 36%, 20% to 34%, 20% to 32%, 20% to 30%, 20% to 28%, 20% to 26%, 20% to 24%, 20% to 22%, 25% to 60%, 25% to 58%, 25% to 56%, 25% to 54%, 25% to 52%, 25% to 50%, 25% to 48%, 25% to 46%, 25% to 44%, 25% to 42%, 25% to 40%, 25% to 38%, 25% to 36%, 25% to 34%, 25% to 32%, 25% to 30%, 25% to 28%, 25% to 26%, 30% to 60%, 30% to 58%, 30% to 56%, 30% to 54%, 30% to 52%, 30% to 50%, 30% to 48%, 30% to 46%, 30% to 44%, 30% to 42%, 30% to 40%, 30% to 38%, 30% to 36%, 30% to 34%, 30% to 32%, 35% to 60%, 35% to 58%, 35% to 56%, 35% to 54%, 35% to 52%, 35% to 50%, 35% to 48%, 35% to 46%, 35% to 44%, 35% to 42%, 35% to 40%, 35% to 38%, 35% to 36%, 40% to 60%, 40% to 58%, 40% to 56%, 40% to 54%, 40% to 52%, 40% to 50%, 40% to 48%, 40% to 46%, 40% to 44%, or 40% to 42% by volume based on the total brain volume, but is not limited thereto.

In one embodiment of the present invention, a general motor impairment symptom may refer to a decrease in total distance traveled in an open field test (OFT), but is not limited thereto.

In one embodiment of the present invention, a sensorimotor impairment symptom may refer to an increased adhesive removal latency in an adhesive removal test (ART), but is not limited thereto.

In one embodiment of the present invention, a long-term cognitive memory impairment symptom may include an increase in the distance traveled to reach a target (which may be used interchangeably with a target location but is not limited thereto) or an increase in the time taken to reach a target, but is not limited thereto.

In one embodiment of the present invention, the death of hippocampal cells may refer to apoptosis, but is not limited thereto.

In one embodiment of the present invention, the arrangement atrophy of hippocampal cells may refer to a state in which the volume of normal hippocampal cells is partially or completely reduced, but is not limited thereto.

In the present invention, cerebral artery occlusion may be a sudden blockage of a cerebral blood vessel, causing severe neurological disorders such as hemiplegia and decreased consciousness, but is not limited thereto.

In one embodiment of the present invention, the cerebral artery occlusion may be middle cerebral artery occlusion (MCAO), but is not limited thereto. In addition, in one embodiment of the present invention, the cerebral artery occlusion may be induced by performing the MCAO method (Longa E Z, Weinstein P R, Carlson S, Cummins R: Reversible middle cerebral artery occlusion without craniectomy in rats, Stroke 20:84-91, 1989), but is not limited thereto.

In the present invention, the cerebral artery is an artery that supplies blood to the cerebrum, and may be classified into the anterior cerebral artery that supplies blood to the front part of the cerebrum, the middle cerebral artery, and the posterior cerebral artery, but is not limited thereto.

In the present invention, when the term “comprising” is used, this does not mean excluding other components unless otherwise specified, but rather that other components may be further included. The terms “step of doing ˜” or “step of ˜” used throughout the present specification do not mean “step for ˜.”

In one embodiment of the present invention, the mouse model was divided into a healthy wild-type control group and an ischemic stroke experimental group, and behavioral tests and tissue analyses were performed, but the analysis is not limited thereto. Specifically, the MCAO mouse model may be produced by a method of occluding the MCA without a craniectomy, and furthermore, the process of experiencing reperfusion after occlusion of the MCA mimics the ischemic cascade experience environment of an actual human acute ischemic stroke patient, thereby providing a similar cerebral infarction lesion area. Therefore, research on neuronal damage and long-term cognitive memory impairment may be conducted using the MCAO model of the present invention, but is not limited thereto.

In one embodiment of the present invention, the hippocampus may be involved in the formation of long-term cognitive memory impairment as a member of the limbic system. In addition, in one embodiment of the present invention, it may be confirmed that the increase or decrease of such hippocampal biomarkers is significant in relation to the severity of post-stroke cognitive impairment. As a result, it may be confirmed through specific examples that Prox1 or Dcx may be utilized as a biomarker for novel hippocampal damage and long-term cognitive memory impairment, but is not limited thereto.

In the present invention, measurement may have a meaning that encompasses detecting and confirming the presence (expression) of a target substance, or detecting and confirming a change in the presence level (expression level) of a target substance. The measurement may be performed without limitation, including both qualitative methods (analysis) and quantitative methods. The types of qualitative methods and quantitative methods for measuring the presence of a substance of the present invention are well known in the art, and the experimental methods described herein may be included therein.

In the present invention, analysis may preferably refer to “measurement,” the qualitative analysis may refer to measuring and confirming the presence or absence of a target substance, and the quantitative analysis may refer to measuring and confirming a change in the presence level (expression level) or amount of a target substance. In the present invention, the analysis or measurement may be performed without limitation, including both qualitative methods and quantitative methods, and preferably, quantitative measurement may be performed.

In one embodiment of the present invention, the expression level of the protein may be measured by any one or more methods selected from the group consisting of Western blot, ELISA, radioimmunoassay (RIA), radioimmunodiffusion, Ouchterlony immunodiffusion, rocket immunoelectrophoresis, immunohistochemistry (IHC), immunoprecipitation assay, complement fixation assay, flow cytometry (fluorescence activated cell sorter (FACS)), and protein chips, and the expression level of the gene may be measured by any one or more methods selected from the group consisting of RT-PCR, competitive RT-PCR, real-time quantitative RT-PCR, multiplex reverse transcription polymerase chain reaction (Multiplex PCR), real-time quantitative reverse transcription polymerase chain reaction (qRT-PCR), RNase protection methods, Northern blotting, DNA chip technology assay, methylated DNA binding domain sequencing (MBDseq) analysis, and reduced representation bisulfite sequencing (RRBS) analysis, but is not limited thereto.

In the present invention, the expression level of a gene may be used interchangeably with the mRNA level of the gene, but is not limited thereto.

In the present invention, a biological sample may be included without limitation as long as it is collected from a subject for diagnosing stroke or post-stroke neurological damage or diagnosing the severity thereof or predicting stroke prognosis, but preferably, it may be a tissue with a lesion. In the present invention, the tissue may be a stroke tissue or cell, but is not limited thereto.

In one embodiment of the present invention, the biological sample may be any one selected from the group consisting of neurons, neural tissue, blood, serum, whole blood, plasma, urine, saliva, cells, tissues, organs, saliva, urine, stool, bone marrow, fine needle aspiration specimens, core needle biopsy specimens, and vacuum aspiration biopsy specimens, but is not limited thereto.

The biological sample may be pretreated before it is to be used for detection or diagnosis. For example, the pretreatment may include homogenization, filtration, distillation, extraction, concentration, inactivation of interfering ingredients, addition of reagents, and the like. The sample may be prepared to increase the detection sensitivity of a protein marker, for example, a sample obtained from a subject may be pretreated using methods such as anion exchange chromatography, affinity chromatography, size exclusion chromatography, liquid chromatography, sequential extraction, or gel electrophoresis.

In one embodiment of the present invention, a control biological sample may be a biological sample isolated from a normal subject or a subject with a mild stroke, but is not limited thereto.

In one embodiment of the present invention, the severity of stroke or post-stroke neurological damage may be high when the cerebral infarction area is 10% to 60%, 10% to 58%, 10% to 56%, 10% to 54%, 10% to 52%, 10% to 50%, 10% to 48%, 10% to 46%, 10% to 44%, 10% to 42%, 10% to 40%, 10% to 38%, 10% to 36%, 10% to 34%, 10% to 32%, 10% to 30%, 10% to 28%, 10% to 26%, 10% to 24%, 10% to 22%, 10% to 20%, 15% to 60%, 15% to 58%, 15% to 56%, 15% to 54%, 15% to 52%, 15% to 50%, 15% to 48%, 15% to 46%, 15% to 44%, 15% to 42%, 15% to 40%, 15% to 38%, 15% to 36%, 15% to 34%, 15% to 32%, 15% to 30%, 15% to 28%, 15% to 26%, 15% to 24%, 15% to 22%, 15% to 20%, 20% to 60%, 20% to 58%, 20% to 56%, 20% to 54%, 20% to 52%, 20% to 50%, 20% to 48%, 20% to 46%, 20% to 44%, 20% to 42%, 20% to 40%, 20% to 38%, 20% to 36%, 20% to 34%, 20% to 32%, 20% to 30%, 20% to 28%, 20% to 26%, 20% to 24%, 20% to 22%, 25% to 60%, 25% to 58%, 25% to 56%, 25% to 54%, 25% to 52%, 25% to 50%, 25% to 48%, 25% to 46%, 25% to 44%, 25% to 42%, 25% to 40%, 25% to 38%, 25% to 36%, 25% to 34%, 25% to 32%, 25% to 30%, 25% to 28%, 25% to 26%, 30% to 60%, 30% to 58%, 30% to 56%, 30% to 54%, 30% to 52%, 30% to 50%, 30% to 48%, 30% to 46%, 30% to 44%, 30% to 42%, 30% to 40%, 30% to 38%, 30% to 36%, 30% to 34%, 30% to 32%, 35% to 60%, 35% to 58%, 35% to 56%, 35% to 54%, 35% to 52%, 35% to 50%, 35% to 48%, 35% to 46%, 35% to 44%, 35% to 42%, 35% to 40%, 35% to 38%, 35% to 36%, 40% to 60%, 40% to 58%, 40% to 56%, 40% to 54%, 40% to 52%, 40% to 50%, 40% to 48%, 40% to 46%, 40% to 44%, or 40% to 42% by volume based on the total brain volume, but is not limited thereto.

In the present invention, in Step (b), the expression level of the protein or the gene being higher than that of the control biological sample means that what was not detected has been detected, or that the detected amount is relatively more than the normal level. For example, the level being “high” means that the level of the experimental group is at least 1%, 2%, 3%, 4%, 5%, 10% or more, for example, 5%, 10%, 20%, 30%, 40%, or 50%, 60%, 70%, 80%, 90%, or more higher than that of the control group, and/or 0.5 times, 1.1 times, 1.2 times, 1.4 times, 1.6 times, 1.8 times, or more higher. Specifically, it may mean, an increase of 1 to 1.5 times, 1.5 to 2 times, 2 to 2.5 times, 2.5 to 3 times, 3 to 3.5 times, 3.5 to 4 times, 4 to 4.5 times, 4.5 to 5 times, 5 to 5.5 times, 5.5 to 6 times, 6 to 6.5 times, 6.5 to 7 times, 7 to 7.5 times, 7.5 to 8 times, 8 to 8.5 times, 8.5 to 9 times, 9 to 9.5 times, 9.5 to 10 times, or more than 10 times compared to that of the control group, but is not limited thereto. The meaning of the opposite term thereof may be understood by those skilled in the art as having the opposite meaning according to the above definition.

Furthermore, the present invention provides is a method of treating stroke or post-stroke neurological damage, including:
(a) measuring the expression level of one or more mRNAs selected from the group consisting of Prox1 and Dcx from a biological sample isolated from a subject;
(b) comparing the expression level of the mRNAs with that of a control biological sample;
(c) determining that the severity of stroke or post-stroke neurological damage is high when the mRNA level of Prox1 is at least 2.03±0.64 as measured in Step (b); or
determining that the severity of stroke or post-stroke neurological damage is high when the mRNA level of DCX is at least 1.63±0.58 as measured in Step (b); and
(d) treating the subject determined to have high severity of stroke or post-stroke neurological damage in Step (c) by administering a pharmaceutically effective amount of a preparation for treating the subject.

Furthermore, the present invention provides is a method of treating stroke or post-stroke neurological damage, including:
(a) measuring the expression level of one or more mRNA selected from the group consisting of Prox1 and Dcx or a gene encoding the protein from a biological sample isolated from an individual;
(b) comparing the expression level of the mRNA with that of a control biological sample;
(c) determining that the subject has a stroke or post-stroke neurological damage when the mRNA level of Prox1 is at least 2.03±0.64 as measured in Step (b); or
determining that the subject has a stroke or post-stroke neurological damage when the mRNA level of DCX is at least 1.63±0.58 as measured in Step (b); and
(d) treating the subject determined to have a stroke or post-stroke neurological damage in Step (c) by administering a pharmaceutically effective amount of a preparation for treating the subject.

In the present invention, “administration” refers to providing a predetermined pharmaceutically effective amount of preparation for treating the target disease to a subject by any appropriate method.

In the present invention, the subject refers to a subject that is in need of risk prediction, diagnosis, prognosis prediction, or treatment of a disease and more specifically, may refer to a mammal such as a human or non-human primate, mouse, rat, dog, cat, horse, and cow, but is not limited thereto. In addition, in one embodiment of the present invention, the subject may be a normal subject, a subject with a stroke, or a subject with post-stroke neurological damage, but is not limited thereto. In the present invention, a subject may be used interchangeably with an animal model, but is not limited thereto.

The term “treatment” or “treating” refers to a decrease in the symptoms associated with the disorder or an amelioration of the recurrence of the symptoms of the disorder, prophylaxis, or reversal of a disease or disorder, or at least one discernible symptom thereof. In the present invention, treatment refers to any action that ameliorates or beneficially changes a target disease and the resulting metabolic abnormalities, and methods such as chemotherapy, surgical operations, or biological therapy may be used.

Hereinafter, preferred examples are presented to help understand the present invention. However, the following examples are provided only to help understand the present invention more easily, and the content of the present invention is not limited by the following examples.

### [EXAMPLES] MATERIALS AND METHODS

### Example 1. Materials

An Ifran solution (isoflurane), an anesthetic used for mouse inhalation anesthesia, was from Hana Pharmaceutical Co. Ltd. HUAYON (Cat No. 14-0203) was used as an MCAO suture. Silk (6-0) was used as a suture. In the RNA extraction process, easy-BLUE total RNA extraction kit (Trizol, 17061) from iNtRON Biotechnology Inc., chloroform (C2432-500ML) and pure ethyl alcohol (E7023-500ML) from SIGMA-ALDRICH, and isopropanol (19516-500ML) from SIGMA were used. All-In-One 5× cDNA Master Mix (CellScript) was used as a reverse transcriptase. TOPreal SYBR Green qPCR PreMIX (Enzynomics Co. Ltd., RT500M) was used as a real-time polymerase chain reaction (RT-PCR) dye.

### Example 2. Laboratory Animals and Study Design

All experiments were performed after receiving the approval of the Institutional Animal Care and Use Committee (IACUC) (SCH22-0097). The rearing space of mice used in the experiment had a 12-hour day/night cycle (7:00 a.m. to 7:00 p.m.), and the indoor temperature was maintained at 23° C.±1° C. and the indoor humidity was maintained at 50%+5%.

### Example 3. Laboratory Animals and Classification Criteria

The laboratory animals used in the study were house mice (Mus musculus) supplied by Raonbio Co. Ltd. (Yongin, Korea). All mice were male of the C57BL/6J strain, weighed 25 g, aged 8 weeks, and randomly assigned to groups. The groups were designed to be a healthy wild-type (WT, n=6) control group, a low-severity MCAO1 (n=6) group and a high-severity MCAO2 (n=6) group based on the neurological score after ischemic stroke. The classification criteria were the most prominent neurological damage confirmed at 48 hours after MCAO. Therefore, individuals with a 48-hour neurological score of less than four points were classified into MCAO1, that is, the low-damage group, and individuals with a neurological score of four points or more were classified into MCAO2, that is, the high-damage group.

### Example 4. MCAO Treatment Method

Longa's MCAO method was performed with the experimental group to implement a mouse model with sequelae after ischemic stroke (Longa E Z, Weinstein P R, Carlson S, Cummins R: Reversible middle cerebral artery occlusion without craniectomy in rats. Stroke 20:8 4-91, 19 8 9). Through the blood flow measurement using noninvasive laser Doppler (OxyFlo™ Pro, OXFORD OPTRONIX) at three stages of preoperative, intraoperative, and postoperative stages, normal blood flow, blood flow during MCAO, and blood flow after achieving reperfusion were measured.

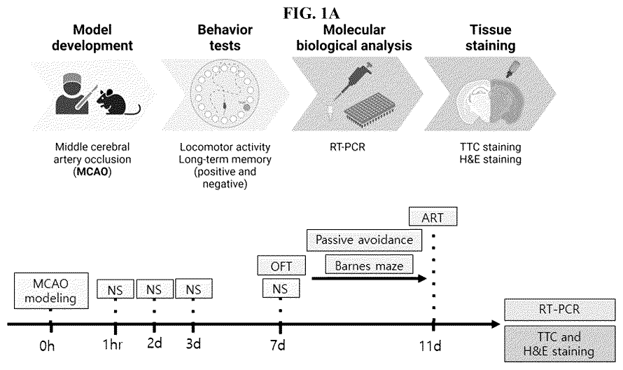

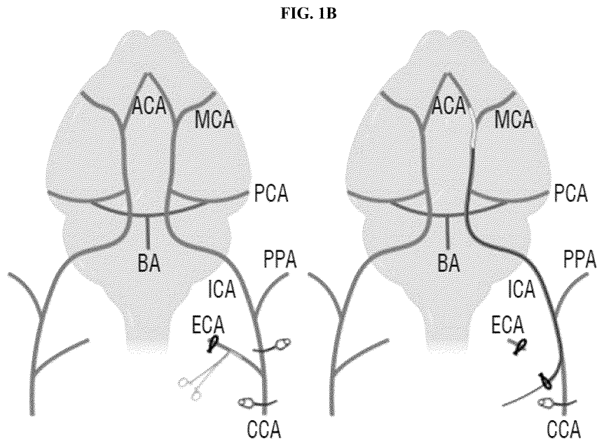

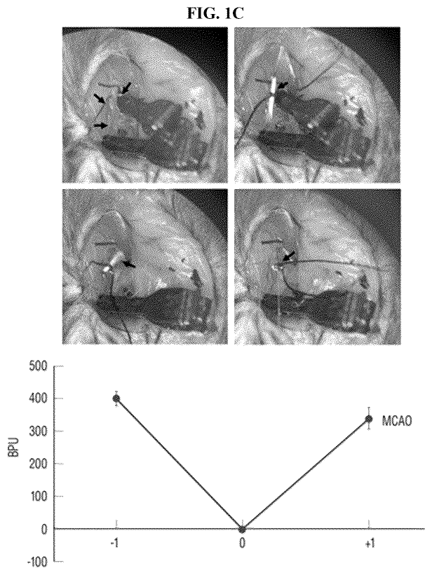

The mice were placed in an isoflurane inhalation anesthesia device, anesthesia was induced with 2.5% isoflurane, and after the mice became motionless, they were transferred to the operating table, where the anesthesia concentration was reduced to 1.5% isoflurane, and surgical treatment was performed. A heating pad maintained at 37° C. was placed on the operating table to prevent the mouse from losing its body temperature. Using surgical scissors, the neck of the mouse was incised vertically (in the shape of the letter ‘I’) to secure a field of vision to view the thymus. When the Y-shaped tissue-overlapping area next to the thymus was spread with forceps to perform tissue dissection, the bifurcation point of the common carotid artery (CCA), the external carotid artery (ECA), and the internal carotid artery (ICA) were confirmed in a Y shape within the field of vision, and the vagus nerve was confirmed as a white solid line near the ICA. Once the position of the vessels was confirmed, vessel clamping was performed on the CCA and ICA to block blood flow (FIG. 1B). After permanent ligation was performed using a suture at the upper end of the ECA to confirm that all blood flow in the Y zone was stopped, a suture was inserted below the ECA and a knot was tied in advance, and an incision was made between the upper ligation portion and the lower knot portion just enough to fit the head of the MCAO suture, and the MCAO suture was inserted from top to bottom. Thereafter, the lower knot portion was tightly tied to prevent the MCAO suture from being released, and then the vessel to which the MCAO suture was fixed was cut just below the ligation portion of the ECA to allow free movement. The vessel clamp on the ICA was released, and the MCAO suture was slowly advanced upward from the ICA to the position where the limit line of the MCAO suture was marked. When the limit line was reached, the filament head of the MCAO suture reached the M1 portion of the MCA, thereby blocking blood flow to the MCA. After performing vessel clamping on the suture insertion portion to fix the suture so as not to be released, ischemic damage was maintained for 30 minutes.

After 30 minutes, the suture was withdrawn to achieve reperfusion. After the suture was removed, the fixing knot was tightened to completely close the incision portion of the ECA. After disinfecting and washing the surgically treated portion with 70% ethanol and phosphate-buffered saline (PBS), the thymus was covered again, and the incision portion was sutured using silk (6-0, silk). After the treatment, the mice were isolated in a recovery cage, and their body temperature was maintained through an infrared heat irradiator.

### Example 5. Neurological Score Measurement Method after MCAO Treatment

The mice that woke up from anesthesia and were confirmed to be active postoperatively were returned to the cages of the same groups and provided with recovery food. Thereafter, the neurological score was measured to determine the level of sequelae after the ischemic stroke and the health status of the mice. The measurement was performed on all the mice, and the measurement range included two measurement items: torso bending and forelimb grip strength. The measurement timepoints were immediately after waking up from anesthesia, and on days 1, 2, 3, and 7 after the MCAO. Torso bending was measured by lifting the mouse by holding its tail and measuring the degree to which the body of the mouse bent in the air in the opposite direction to the side where hemiplegia was induced. Three points were given for an unmeasurable degree, two points were given for severe body bending, one point was given for mild body bending, and 0 points were given when there was no problem in the motion. Forelimb grip strength was measured by having the mice grip a horizontal bar with both forelimbs and measuring the point at which the forelimb with hemiplegia fell prior to the other forelimb. Three points were given when the falling point was unmeasurable, two points were given when the forelimb with induced hemiplegia fell much faster than the other forelimb, one point was given when the forelimb fell slowly due to mild hemiplegia, and zero points were given when both forelimbs had no problem in gripping the horizontal bar. The scores for torso bending and grip strength were added together, and a grade was given from 0 to a maximum of 6 points to each individual.

### Example 6. Experimental Method of Behavioral Analysis

Each of the individuals underwent a behavioral analysis to analyze behavioral changes such as activity, emotion, and cognitive memory due to sequelae after ischemic stroke. The behavioral analysis began on day 7 after MCAO treatment, and considering the continuous stress received by the mice, the experiment was designed in the order of low to high external stimuli and conducted for a total of five days (day 11 after MCAO treatment) including the adaptation period. Each behavioral experiment was video-recorded for analysis.

### Example 6.1. OFT Method

A cubic transparent acrylic box (45*45*45 cm, x*y*z) with an open top was divided into a central zone corresponding to 50% of the horizontal (x) and vertical (y) axes and a peripheral zone. The mice were placed in the central zone and allowed to move freely for ten minutes. After the experiment, the total distance traveled (cm) was analyzed using behavioral analysis software.

### Example 6.2. Barnes Maze Test Method

Eighteen local areas were designated in a circular field with a diameter of 92 cm, and the mice's regular food was randomly placed in one of the areas. The field was divided into quadrants, and each quadrant was allocated 25%. The mice were allowed to explore freely for three minutes, starting at the center of the field, and the experiment was terminated early when they reached the food area before three minutes. After an adaptation period of three days, the time taken to reach the food area (latency to target, sec) and distance (distance to target, cm) were analyzed on the 4th test day.

### Example 6.3. Adhesive Removal Test Method

A single-sided tape (3*2 mm, x*y) was attached to one forefoot of the mouse, and the mouse was placed in a translucent cage without bedding, allowing it to move freely. The mouse was observed for up to two minutes, and the time it took for the mouse to remove the tape (latency L.arm or latency R.arm, see) was analyzed. The measurement was performed with both forefeet, with one side measured first and then the other side was measured.

### Example 7. Camera Tracking Analysis Method

A behavioral analysis experiment was conducted, and the recorded video was analyzed with Smart 3.0 Video Tracking System (Panlab, Barcelona, Spain, hereinafter referred to as Smart 3.0) software. The Smart 3.0 system detected movement by comparing the reference image and the current video. The experimental time, mouse position, distance moved by the mouse in the zone, zone occupancy time by the mouse, and number of times the mouse crossed between zones were precisely and automatically measured to analyze and quantify the behavioral differences between each mouse.

### Example 8. RT-PCR Analysis Method

After completing the behavioral analysis experiment, the group was randomly divided into two types of individuals: individuals for histological analysis and individuals for transcriptome (messenger RNA, mRNA) level analysis.

### Example 8.1. Brain Tissue Extraction and Homogenization Method

After inducing isoflurane inhalation anesthesia, the mice were euthanized by cervical dislocation. After confirming euthanasia, the mice were decapitated with surgical scissors to obtain the head, and the scalp was rolled up into two frontal sections to secure the skull. Forceps were inserted under the dura and carefully lifted outward to remove the bone, and the whole brain tissue was obtained. The obtained brain tissue was transferred to a 2 ml microtube and 1 ml of easy-BLUE (Trizol) was added. Immediately, the brain tissue and the easy-BLUE solution were mixed evenly using a homogenizer.

### Example 8.2. RNA Extraction Method

After adding 200 μl of chloroform to the homogeneously mixed tissue and solution, the centrifuge was operated at 13,000 rpm and 4° C. for ten minutes. After ten minutes, when the protein, DNA, and RNA layers were separated, 400 μl of the transparent supernatant, which was the RNA layer, was obtained and transferred to a 1.5 ml microtube, and then isopropanol was added in an equal volume (1:1) to precipitate the RNA for 10 minutes. After precipitation, centrifugation was performed again at 13,000 rpm and 4° C. for five minutes. After centrifugation, the RNA pellet was confirmed, and only the supernatant was carefully removed. After removal, 1 ml of ethanol diluted in 75% nuclease free water (NFW) was added to wash away the remaining solvent. After centrifugation again at 10,000 rpm and 4° C. for five minutes, the supernatant was removed and the pellet was dried until no moisture was left. 20 μl of preheated NFW was added, and the amount of RNA was quantified as 1000 ng/mL using NanoDrop, and all individuals were calibrated equally.

### Example 8.3. Complementary DNA (cDNA) Synthesis Method

According to the standard protocol provided by All-In-One 5× cDNA Master Mix (CellScript, Madison, WI, USA), NEW and 2 μl of the master mix were added to the calibrated and diluted RNA solution to adjust the final volume to 10 μl. After the addition, cDNA synthesis through reverse transcription was performed according to the cDNA synthesis standard protocol provided by T100 Thermal Cycler (BIO-RAD, Contra Costa, CA, USA). After the synthesis was completed, 90 μl of NEW was added to dilute the cDNA 1/10.

### Example 8.4. RT-PCR Method

Primers corresponding to the transcript to be targeted, SYBR green, and NFW were dispensed into a well of a 96-well plate in a volume of 14 μl according to the standard protocol, and 1 μl of cDNA for each individual was added to adjust the total volume to 15 μl. A sealing film for PCR was attached on top of the 96-well plate to prevent the contents of each well from evaporating or external substances from entering. The plate was mounted on the CFX96 Real-Time PCR System (BIO-RAD), and the polymerase reaction was performed according to the PCR protocol. The PCR protocol included heating at 95° C. for 15 minutes and then 40 cycles of 95° C. for 10 seconds, 60° C. for 15 seconds, and 72° C. for 20 seconds. The primer sequences corresponding to the transcripts used in RT-PCR are shown in Table 3 below.

TABLE 3

| | Classi- | | Sequence ID |
|---|---|---|---|
| Gene | fication | Sequence | number |
| Ccr2 | Front | 5′-TCC ACT CTA CTC CCT GGT ATT C-3′ | SEQ ID NO: 5 |
| | Rear | 5′-TGG CCA AGT TGA GCA GAT AG-3′ | SEQ ID NO: 6 |
| Dcx | Front | 5′-AGG GAG TGC GCT ACA TTT ATA C-3′ | SEQ ID NO: 7 |
| | Rear | 5′-GTT GTC TGA GGA GCA GAC ATA G-3′ | SEQ ID NO: 8 |
| Icam1 | Front | 5′-GTG ATG GCA GCC TCT TAT GT-3′ | SEQ ID NO: 9 |
| | Rear | 5′-GGG CTT GTC CCT TGA GTT T-3′ | SEQ ID NO: 10 |
| II-1β | Front | 5′-GTG TGT GAC GTT CCC ATT AG-3′ | SEQ ID NO: 11 |
| | Rear | 5′-TGT CCA TTG AGG TGG AGA G-3′ | SEQ ID NO: 12 |
| Mcp1 | Front | 5′-CAC TCA CCT GCT GCT ACT CA-3′ | SEQ ID NO: 13 |
| | Rear | 5′-CTT CTT GGG GTC AGC ACA GA-3′ | SEQ ID NO: 14 |
| Prox1 | Front | 5′-CAC CAG GGA TTG TGA GCT ATA C-3′ | SEQ ID NO: 15 |
| | Rear | 5′-ACG TGA TCT GCG CAA CTT-3′ | SEQ ID NO: 16 |
| Vcam1 | Front | 5′-GCA CTC TAC TGC GCA TCT T-3′ | SEQ ID NO: 17 |
| | Rear | 5′-CAC CAG ACT GTA CGA TCC TTT C-3′ | SEQ ID NO: 18 |

### Example 9: Triphenyltetrazolium Chloride (TTC) Staining Method

After euthanizing the mice, the brain tissue extraction process for RNA extraction was performed in the same manner as before homogenization. Complete brain tissue was obtained and placed in a brain matrix, and brain tissue sections were obtained at 2-mm intervals by cutting with a razor blade to fit the frame. The obtained brain tissue sections were completely immersed in a 2% diluted TTC solution, wrapped in foil, and incubated in an incubator at 37° C. for 30 minutes. After incubation, the percentage (%) of the unstained area (white) in the total tissue area was measured using Image J software (National Institutes of Health, Bethesda, MD, USA).

### Example 10: Hematoxylin and Eosin (H&E) Staining

When the mice were anesthetized and became unconscious, an incision was made between the ribs and the abdomen to obtain a view of the heart. When the view was obtained, a needle was inserted into the convex part of the heart to inject PBS via a syringe. When the needle was inserted, PBS was slowly injected to achieve perfusion. After the blood was drained by perfusion, the brain was obtained through the same incision process, and the tissue was fixed by completely immersing it in 4% diluted paraformaldehyde.

The fixed brain tissue was cut to a thickness of approximately 2 to 3 mm and subjected to tissue processing (STP120 Spin Tissue Processor; Especialidades Medicas Y R, L., Tarragona, Spain). The processed tissue was sectioned to a thickness of approximately 3 to 4 μm using a microtome (Finese ME Microtome; Thermo Fisher Scientific, Waltham, MA, USA). Then, the sections were attached to slides, dried, and deparaffinized in xylene for 3 steps/3 minutes, hydrated in alcohol for 4 steps/2 minutes, in hematoxylin for 10 minutes, in water for 3 minutes, in eosin for 1 minute and 40 seconds, hydrated in alcohol for 4 steps/1 minute, and cleared in xylene for 3 steps/3 minutes. Then, the sections were sealed to prepare H&E specimens.

### Example 11. Statistical Analysis

Quantitative data obtained through behavioral analysis, mRNA level analysis, and TTC staining was graphed and statistically analyzed using GraphPad Prism 8 software (GraphPad Software, Boston, MA, USA). The significance of statistical differences between each control group and experimental group was confirmed using one-way analysis of variance.

### [EXPERIMENTAL EXAMPLES] RESULTS

### Experimental Example 1. Confirmation of Ischemic Stroke Induced by Cerebral Blood Flow Blockage Due to MCAO

The MCAO group, which was the MCAO experimental group, was subjected to reperfusion after Longa MCAO (FIG. 1C). The blood flow was measured by noninvasive laser Doppler just before occlusion of the M1 region of the MCA, and an average blood flow of 400 BPU was confirmed. During the MCAO, the blood flow was 0 BPU, and when filament withdrawal and blood reperfusion were achieved, a blood reperfusion between 300 BPU and 400 BPU was confirmed.

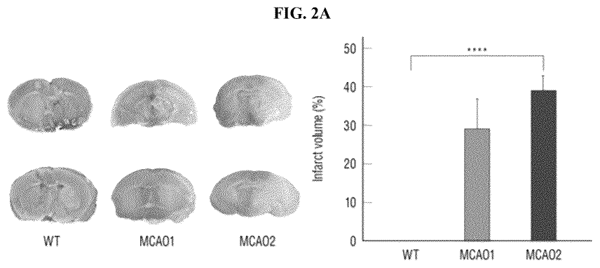

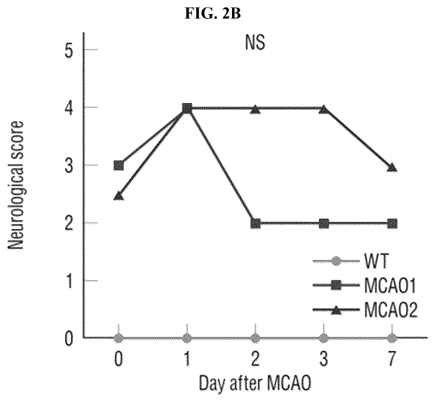

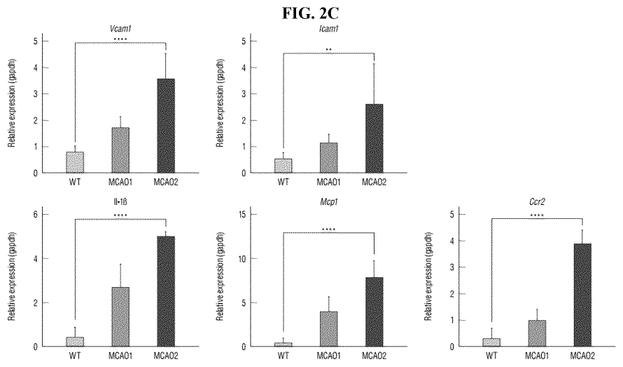

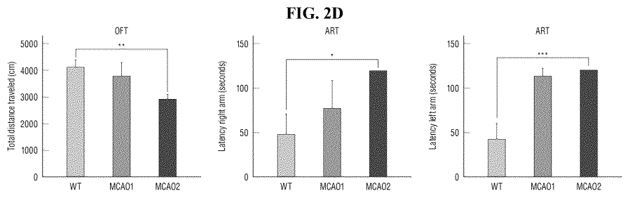

For all groups, after the behavioral analysis was completed, brain tissues obtained after sacrifice were subjected to TTC staining to measure the cerebral infarction area, and cerebral infarction was confirmed in the MCAO1 and MCAO2 groups (FIG. 2A, left diagram). No cerebral infarction was confirmed in WT (0%), while MCAO1 and MCAO2 showed infarction areas of 29.02%+7.74% and 38.94%+3.74%, respectively, indicating the MCAO2 had the largest infraction area (FIG. 2A, right diagram) (p<0.0001). MCAO2 showed a higher infarction volume than MCAO1 (p=0.0065). MCAO2 also obtained a high score in the neurological score measurement, confirming a damaged brain (FIG. 2B).

This confirmed that cerebral infarction developed through the same mechanism as human stroke in the MCAO group designed to mimic human ischemic stroke. The cerebral infarction area of the WT group and the MCAO1 and 2 groups was measured through TTC staining, and the MCAO1 and MCAO2 groups developed an infarction area that amounted to 30% of the total brain volume, whereas the WT group exhibited no brain damage area. In addition, through the state where filament withdrawal and blood reperfusion were achieved after MCAO treatment, the state where vascular reperfusion is achieved through a thrombolytic agent or mechanical thrombectomy after the occurrence of ischemic stroke, that is, the pathological state that causes sequelae after ischemic stroke was identically implemented.

In addition, in the MCAO1 and MCAO2 groups that exhibited brain infarction areas, deteriorated body control ability was confirmed through the neurological score measurement. Specifically, as a result of measuring left or right trunk rotation through torso bending, not only was the trunk bending biased to one side due to hemiplegia confirmed, but also permanent right-sided paralysis was confirmed as a sequela in the mice that had experienced ischemic stroke in the left brain. In addition, it was confirmed in the MCAO1 and MCAO2 groups, where grip strength was measured when the mice pulled the horizontal bar with both forelimbs, that the grip strength of the right forelimb was significantly reduced and so the mice were unable to grab the horizontal bar.

As a result, it is suggested that brain cell damage caused by cerebral infarction in MCAO1 and MCAO2 groups also affected body control through nerves.

### Experimental Example 2. Confirmation of the Development of Neuroinflammatory Brain Damage Due to Ischemic Cascade in MCAO Groups

Neuroinflammatory damage due to the ischemic cascade was confirmed in the MCAO groups that experienced ischemic stroke (FIG. 2C).

As a result of RT-PCR performed to confirm the difference in mRNA expression levels, it was found that the mRNA level of vascular cell adhesion molecule 1 (Vcam1), which contributes to immune cell adhesion, was 0.81±0.22 in WT, 1.74±0.21 in MCAO1, and 3.61±0.95 in MCAO2 (p<0.0001). The mRNA level of Vcam1 in MCAO2 was higher than that in MCAO1 (p=0.0013).

The mRNA level of intercellular adhesion molecule 1 (Icam1), which contributes to immune cell adhesion, was 0.54±0.24 in WT, 1.15±0.33 in MCAO1, and 2.63±1.53 in MCAO2 (p=0.0037). The mRNA level of Icam1 in MCAO2 was higher than that in MCAO1 (p=0.0423).

The mRNA level of inflammatory cytokine IL-1ß was 0.44±0.44 in WT, 2.69±1.06 in MCAO1, and 5.02±0.20 in MCAO2 (p<0.0001). The mRNA level of IL-1ß in MCAO2 was higher than that in MCAO1 (p=0.0004).

The mRNA level of monocyte chemoattractant protein 1 (Mcp1), a chemokine receptor that recruits monocytes to inflammation sites, was 0.48±0.50 in WT, 3.98±1.72 in MCAO1, and 7.86±1.85 in MCAO2 (p<0.0001). The Mcp1 mRNA level in MCAO2 was higher than that in MCAO1 (p=0.0038).

The mRNA level of C-C chemokine receptor 2 (Ccr2), which is involved in monocyte infiltration in inflammatory diseases, was 0.33±0.37 in WT, 1.02±0.39 in MCAO1, and 3.89±0.51 in MCAO2 (p<0.0001). The Ccr2 mRNA level in MCAO2 was higher than that in MCAO1 (p=0.0001).

The development of symptoms of cerebral infarction and hemiplegia appears to be caused by ischemic damage to the brain due to the ischemic cascade after stroke onset, and the same was also observed in human ischemic stroke. The lack of oxygen and energy supply due to ischemic conditions caused mitochondrial death and apoptosis. In this process, immune cells including monocytes are guided to the site of apoptosis, causing an inflammatory response at the site where apoptosis occurred.

In Example 2, it was confirmed that the expression of Vcam1 and Icam1, which are adhesion molecules that help the migration of monocytes, and the like, was increased in the MCAO1 and MCAO2 groups, and high mRNA expression levels of Il-1β, Mcp1, and Ccr2, which are inflammation-related chemotactic chemokines and cytokines, were confirmed in the MCAO1 and MCAO2 groups.

Considering what is described above, it is suggested that neurological damage due to the post-stroke ischemic cascade causes the development of cerebral infarction and hemiplegia symptoms, that is, the development of neuroinflammatory brain damage.

### Experimental Example 3. Confirmation of the Development of General Motor Impairment and Sensorimotor Impairment Symptoms in MCAO Groups

The MCAO groups, which suffered from sequelae after ischemic stroke, exhibited general motor impairment symptoms (FIG. 2D). The total distance traveled (cm) in the open field test (OFT) was 4102±621 cm in WT, 3771±490 cm in MCAO1, and 2908±188 cm in MCAO2 (p=0.0015). The total distance of MCAO2 was shorter than that of MCAO1 (p=0.0024).

In the ART for sensorimotor measurement, the latency in the right forefoot latency measurement (Latency R.arm) region was 49±23 sec in WT, 88±45 sec in MCAO1, and 120±0 sec in MCAO2 (p=0.0168). The right forefoot adhesive removal latency was longer in MCAO2 than in WT (p=0.0055).

The latency in the left forefoot latency measurement (Latency L.arm) region was 43±17 sec in WT, 114±9 sec in MCAO1, and 120±0 sec in MCAO2 (p=0.0004). There was a statistical difference between WT and MCAO1 (p=0.0019), and the left forefoot adhesive removal latency was longer in MCAO2 than in WT (p=0.0011).

As a result, the behavioral analysis confirmed behavioral disorders in the MCAO1 and MCAO2 groups. In the OFT, MCAO1 and MCAO2 groups showed less activity than the WT group. In addition, in the ART, MCAO1 and MCAO2 groups had difficulties in removing the tape on both forefeet compared to the WT group, reaching a maximum latency. This suggests that MCAO1 and MCAO2 groups exhibited symptoms of general motor and sensorimotor disorders.

### Experimental Example 4. Confirmation of Occurrence of Long-Term Cognitive Memory Impairment in the MCAO Groups

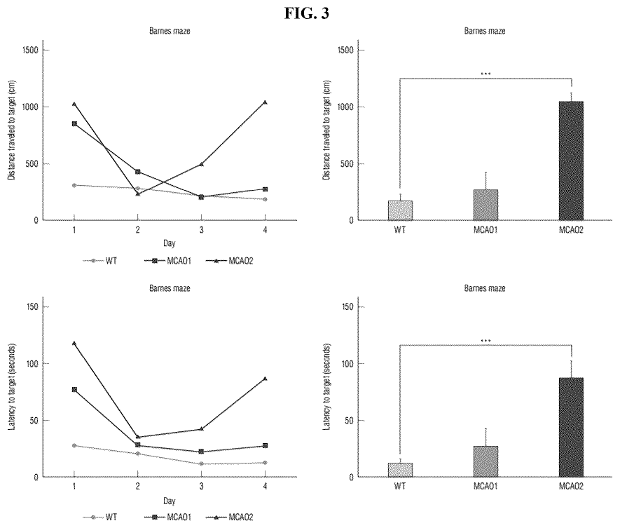

The MCAO groups, which suffered from sequelae after ischemic stroke, developed positive long-term cognitive memory impairment (FIG. 3). The distance to target (cm) was 178±131 cm in WT, 276±216 cm in MCAO1, and 1051±102 cm in MCAO2 (p=0.0004). The distance to target was longer in MCAO2 than in MCAO1 (p=0.0442).

In terms of latency to target (seconds), WT showed a latency of 13.3±7.5 seconds, MCAO1 showed a latency of 27.9±21.1 seconds, and MCAO2 showed a latency of 87.9±21.2 seconds (p=0.0007). MCAO2 showed a longer latency to target than WT (p=0.0002).

As a result, in the Barnes maze test to confirm positive long-term recognition memory, the MCAO1 and MCAO2 groups traveled a longer moving distance and took a longer latency to reach the target from the first day, and experienced many errors on the last measurement day of the analysis, traveling a longer moving distance and taking a longer latency. On the other hand, the WT group traveled a very short moving distance and took a shorter latency. This suggests that impairment after ischemic stroke causes long-term recognition memory impairment.

### Experimental Example 5. Changes in mRNA Activity of Prox1 and Dcx and Confirmation of Neurological Damage in the MCAO Groups

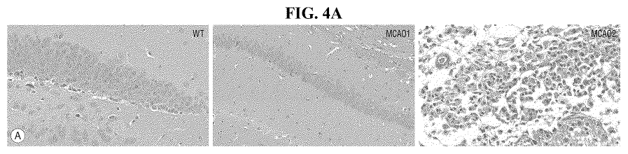

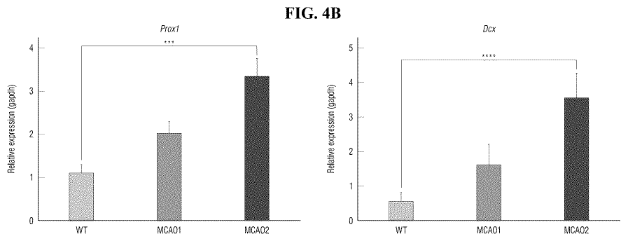

In the MCAO groups, neuronal damage was confirmed (FIG. 4A). In the healthy WT, the hippocampal cell arrangement was even, and no apoptosis was confirmed, but in MCAO1, hippocampal cell arrangement atrophy was confirmed although no apoptosis was confirmed, and in MCAO2, extensive apoptosis was confirmed.

The mRNA level of Prox1 was confirmed to be higher in MCAO2 than in MCAO1 (FIG. 4B, left diagram). The mRNA level was 1.12±0.46 in WT, 2.03±0.64 in MCAO1, and 3.36±1.01 in MCAO2 (p=0.0004). The mRNA expression level of Prox1 was confirmed to be higher in MCAO2 than in MCAO1 (p=0.0215).

The mRNA level of Dcx, a nerve regeneration marker, was confirmed to be higher in MCAO2 than in MCAO1 (FIG. 4B, right diagram). The mRNA level was 0.59±0.24 in WT, 1.63±0.58 in MCAO1, and 3.57±0.71 in MCAO2 (p<0.0001). The mRNA expression level of Dcx was confirmed to be higher in MCAO2 than in MCAO1 (p=0.0004).

### Experimental Example 6. Confirmation that Increased mRNA Levels of Prox1 and Dcx can be Used as Indicators of Neurological Damage

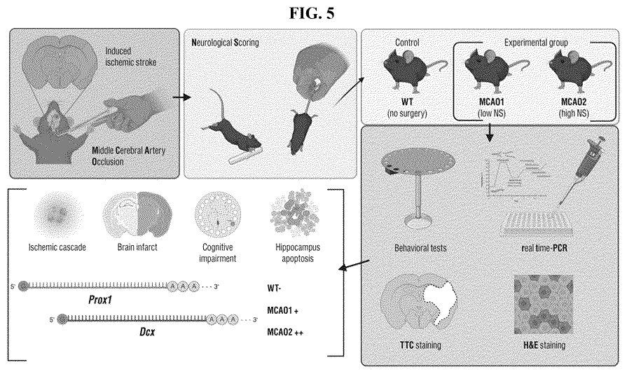

It was confirmed that increased mRNA expression levels of Prox1 and Dcx can be used as indicators of neurological damage (FIG. 5). Among behavioral disorders after ischemic stroke, there is long-term cognitive memory impairment. The expression of Prox1 was the highest in the severe MCAO2 group compared to the MCAO1 group, and brain tissue damage was confirmed through tissue H&E staining to confirm neurological damage, and consistent results were also found in the Barnes maze test. This suggests that Prox1 and Dcx can be utilized as new predictors of neurological damage.

In the present invention, it was found that MCAO2 with high severity had the highest mRNA expression level of Prox1. This suggests that the increased mRNA level expression of Prox1 is caused by hippocampal damage, and this result is consistent with the H&E staining confirming hippocampal apoptosis in the MCAO group. In the present invention, when the degree of neurological damage was compared through tissue staining between MCAO2 with high severity and MCAO1 with low severity, it was confirmed that the expression of Prox1 and Dcx was low in MCAO1, which is consistent with low neurological damage.

The increased mRNA expression levels of Prox1 and Dcx were associated with worsening of motor activity and upregulation of biomarkers related to neuroinflammation. Brain infarction volume was measured using TTC staining in the MCAO groups, and the MCAO2 group, which had the most severe neurological damage, showed the highest brain infarction volume. It seems that the development of symptoms of cerebral infarction and hemiplegia is caused by ischemic damage to the brain due to the post-stroke ischemic cascade, which is also the case in human ischemic stroke. The lack of oxygen and energy supply due to ischemia caused mitochondrial death and apoptosis. Vcam1 and Icam1, adhesion molecules that help the migration of monocytes, were found to be upregulated in the MCAO2 group. Higher mRNA levels of IL-1β, Mcp1, Ccr2, and inflammation-related chemokines and cytokines were found in the MCAO2 group. The behavioral tests confirmed behavioral disorders in the MCAO groups. In the OFT, the MCAO2 group showed lower activity compared to the MCAO1 group. In the ART, the MCAO2 group had more difficulties in removing the tape from both forefeet than the MCAO1 group, reaching a maximum latency. In the Barnes maze test, which confirms positive long-term recognition memory, the difference was more prominent. The MCAO2 group traveled a longer travel distance and took a longer latency to reach the destination than those of the MCAO1 group, from the first day of the preliminary test, and on the day of the final measurement of the analysis, the distance traveled and the latency increased due to more errors.

As a result, this suggests that increased mRNA levels of Prox1 and Dcx may be used as indicators of neurological damage, i.e., for diagnosing stroke or post-stroke neurological damage, for diagnosing the severity of stroke or post-stroke neurological damage, and for predicting stroke prognosis.

Using an ischemic stroke mouse disease model, a tissue damage analysis and a behavioral analysis were performed, and the mRNA level expression were confirmed, and as a result, Prox1 or Dcx could be selected as a biomarker. In addition, it was confirmed that the expression of Prox1 or Dcx increased as the severity of stroke or post-stroke neurological damage was higher, and thus the present invention is expected to be useful for diagnosing stroke or post-stroke neurological damage, for diagnosing the severity of stroke or post-stroke neurological damage, and for predicting stroke prognosis.

The above description of the present invention is for illustrative purposes only, and those skilled in the art will understand that the present invention may be easily modified into other specific forms without changing the technical idea or essential features of the present invention. Therefore, it should be understood that the examples described above are illustrative in all respects and not restrictive.

What is claimed is:

1. A method of diagnosing stroke or post-stroke neurological damage, comprising:
(a) measuring the expression level of one or more mRNAs selected from the group consisting of Prox1 and Dex from a biological sample isolated from a subject;
(b) comparing the expression level of the mRNAs with that of a control biological sample;
(c) determining that the subject has a stroke or post-stroke neurological damage when the mRNA level of Prox1 is at least 2.03±0.64 as measured in Step (b); or
determining that the subject has a stroke or post-stroke neurological damage when the mRNA level of DCX is at least 1.63±0.58 as measured in Step (b); and
(d) treating the subject determined to have a stroke or post-stroke neurological damage in Step (c) by administering a pharmaceutically effective amount of a preparation for treating the subject.

2. The method of claim 1, wherein the stroke is one or more selected from the group consisting of hemorrhagic stroke and ischemic stroke.

3. The method of claim 1, wherein the stroke or post-stroke neurological damage is characterized by any one or more selected from the group consisting of:
(a) occurrence of a symptom of decreased blood flow reperfusion;
(b) occurrence of a symptom of increased cerebral infarction area;
(c) occurrence of a general motor impairment symptom;
(d) occurrence of a sensorimotor impairment symptom;
(e) occurrence of long-term cognitive memory impairment; and
(f) occurrence of death or arrangement atrophy of hippocampal cells.

4. The method of claim 1, wherein the stroke or post-stroke neurological damage is characterized by an increase of mRNA level of any one or more selected from the group consisting of:
vascular cell adhesion molecule 1 (Vcam1), intercellular adhesion molecule 1 (Icam1), interleukin-1 beta (IL-1β), monocyte chemoattractant protein 1 (Mcp1), and C-C chemokine receptor 2 (Ccr2).

5. The method of claim 1, wherein the stroke or post-stroke neurological damage is due to cerebral artery occlusion.

6. The method of claim 5, wherein the cerebral artery is any one or more selected from the group consisting of anterior cerebral artery, middle cerebral artery, and posterior cerebral artery.

7. The method of claim 1, wherein the expression level of the mRNA is measured by any one or more methods selected from the group consisting of RT-PCR, competitive RT-PCR, real-time quantitative RT-PCR, multiplex reverse transcription polymerase chain reaction (Multiplex PCR), real-time quantitative reverse transcription polymerase chain reaction (qRT-PCR), RNase protection methods, Northern blotting, DNA chip technology assay, methylated DNA binding domain sequencing (MBDseq) analysis, and reduced representation bisulfite sequencing (RRBS) analysis.

8. A method of treating stroke or post-stroke neurological damage, comprising:
(a) measuring the expression level of one or more mRNAs selected from the group consisting of Prox1 and Dcx from a biological sample isolated from a subject;
(b) comparing the expression level of the mRNAs with that of a control biological sample;
(c) determining that the severity of stroke or post-stroke neurological damage is high when the mRNA level of Prox1 is at least 2.03±0.64 as measured in Step (b); or
determining that the severity of stroke or post-stroke neurological damage is high when the mRNA level of DCX is at least 1.63±0.58 as measured in Step (b); and
(d) treating the subject determined to have high severity of stroke or post-stroke neurological damage in Step (c) by administering a pharmaceutically effective amount of a preparation for treating the subject.

9. A method of treating stroke or post-stroke neurological damage, comprising:
(a) measuring the expression level of one or more mRNA selected from the group consisting of Prox1 and Dex or a gene encoding the protein from a biological sample isolated from an individual;
(b) comparing the expression level of the mRNA with that of a control biological sample;
(c) determining that the stroke is high when the mRNA level of Prox1 is at least 2.03±0.64 as measured in Step (b); or
determining that stroke is high when the mRNA level of DCX is at least 1.63±0.58 as measured in Step (b); and
(d) treating the subject determined to have a stroke or post-stroke neurological damage in Step (c) by administering a pharmaceutically effective amount of a preparation for treating the subject.

10. The method of claim 9, wherein the stroke is one or more selected from the group consisting of hemorrhagic stroke and ischemic stroke.

11. The method of claim 9, wherein the stroke or post-stroke neurological damage is characterized by any one or more selected from the group consisting of:
(a) occurrence of a symptom of decreased blood flow reperfusion;
(b) occurrence of a symptom of increased cerebral infarction area;
(c) occurrence of a general motor impairment symptom;
(d) occurrence of a sensorimotor impairment symptom;
(e) occurrence of long-term cognitive memory impairment; and
(f) occurrence of death or arrangement atrophy of hippocampal cells.

12. The method of claim 9, wherein the stroke or post-stroke neurological damage is characterized by an increase of mRNA level of any one or more selected from the group consisting of:
vascular cell adhesion molecule 1 (Vcam1), intercellular adhesion molecule 1 (Icam1), interleukin-1 beta (IL-1β), monocyte chemoattractant protein 1 (Mcp1), and C-C chemokine receptor 2 (Ccr2).

13. The method of claim 9, wherein the stroke or post-stroke neurological damage is due to cerebral artery occlusion.

14. The method of claim 13, wherein the cerebral artery is any one or more selected from the group consisting of anterior cerebral artery, middle cerebral artery, and posterior cerebral artery.

15. The method of claim 9, wherein the expression level of the mRNA is measured by any one or more methods selected from the group consisting of RT-PCR, competitive RT-PCR, real-time quantitative RT-PCR, multiplex reverse transcription polymerase chain reaction (Multiplex PCR), real-time quantitative reverse transcription polymerase chain reaction (qRT-PCR), RNase protection methods, Northern blotting, DNA chip technology assay, methylated DNA binding domain sequencing (MBDseq) analysis, and reduced representation bisulfite sequencing (RRBS) analysis.


SEQUENCE LISTING (18 sequences)

SEQ ID NO: 1 - protein, 737 aa, synthetic construct
MPDHDSTALLSRQTKRRRVDIGVKRTVGTASAFFAKARATFFSAMNPQGSEQDVEYSVVQHADGEKSNVLRKLLKRANSYEDAMMPFPGATIISQLLKNNMNKNGGTEPSFQASGLSSTGSEVHQEDICSNSSRDSPPECLSPFGRPTMSQFDVDRLCDEHLRAKRARVENIIRGMSHSPSVALRGNENEREMAPQSVSPRESYRENKRKQKLPQQQQQSFQQLVSARKEQKREERRQLKQQLEDMQKQLRQLQEKFYQVYDSTDSENDEDGDLSEDSMRSEILDARAQDSVGRSDNEMCELDPGQFIDRARALIREQEMAENKPKREGSNKERDHGPNSLQPEGKHLAETLKQELNTAMSQVVDTVVKVFSAKPSRQVPQVFPPLQIPQARFAVNGENHNFHTANQRLQCFGDVIIPNPLDTFGSVQMPSSTDQTEALPLVVRKNSSEQSASGPATGGHHQPLHQSPLSATAGFTTPSFRHPFPLPLMAYPFQSPLGAPSGSFSGKDRASPESLDLTRDTTSLRTKMSSHHLSHHPCSPAHPPSTAEGLSLSLIKSECGDLQDMSDISPYSGSAMQEGLSPNHLKKAKLMFFYTRYPSSNMLKTYFSDVKFNRCITSQLIKWFSNFREFYYIQMEKYARQAINDGVTSTEELSITRDCELYRALNMHYNKANDFEVPERFLEVAQITLREFFNAIIAGKDVDPSWKKAIYKVICKLDSEVPEIFKSPNCLQELLHE

SEQ ID NO: 2 - DNA, 2214 bp, synthetic construct
  source 1..2214: mol_type = other DNA
atgcctgaccatgacagcacagccctcttaagccggcaaaccaagaggagaagggttgacattggagtgaaaaggacggtagggacagcatctgcattttttgctaaggcaagggcaacatttttcagtgccatgaatccccaaggttcagagcaggatgttgaatattctgtggtgcaacacgcagatggggaaaagtcgaacgtactccgcaagctgctgaagagggcgaactcgtatgaagatgccatgatgccttttccaggagcaactataatttcccagctgttgaaaaataacatgaacaaaaacggtggcaccgagcccagtttccaagccagcggactctctagcacaggctccgaagtacatcaggaggatatatgtagcaactcttcaagagacagccccccagagtgtctttccccttttggcaggcctactatgagccagtttgatgtggatcgcttatgtgatgagcacctgagagcaaagcgcgcccgggttgagaatatcattcggggtatgagccattcccccagtgtggcattaaggggcaatgaaaacgaaagagagatggccccgcagtctgtgagtccccgagaaagttacagagaaaacaaacgcaagcagaagctgccccagcagcagcaacagagtttccagcagctggtttcagcccgaaaagaacagaagcgagaggagcgccgacagctgaaacagcagctggaagacatgcagaagcagctgcgccagctgcaggagaagttctaccaggtctatgacagcacagactccgaaaatgatgaagatggcgacctgtctgaagacagcatgcgctcggagatcctggatgcacgggcccaggactcggtggggcgctcagacaatgagatgtgtgagctggacccagggcagttcatcgacagggcccgagccctaatcagggagcaggagatggctgagaacaagcctaagcgagaaggcagcaacaaagaaagagaccacgggccaaactccttgcagccagaaggcaagcatctggcagagaccttaaaacaggagctgaacacggccatgtcgcaggttgtggacacggtggtcaaagtcttctcagccaaaccctctcgccaggttcctcaggtcttcccacctctccagatcccccaggccagattcgcagtcaacggggaaaaccacaatttccacacggccaaccagcgcctgcaatgctttggtgatgtcatcattccgaaccccttggacacctttggcagtgtgcagatgcctagttccacagaccagacggaagcccttcccctggtggtccgaaaaaactcatccgagcaatctgcctctggcccggccactggcggccaccaccagcccctgcaccagtcacccctctccgccactgcaggcttcaccacccctagcttccgccatccctttcccctgcccttgatggcttatccatttcagagtccactaggtgctccctccggctccttctcggggaaggacagagcctctcctgagtccttagacttgactcgggacacaacaagtctgaggaccaagatgtcatcacaccatctgagccaccacccctgttcaccagcacacccacccagcaccgcagaaggactctctttgtcactcataaagtctgagtgtggagatcttcaagatatgtccgacatctcaccttattcaggaagcgcaatgcaggaagggctatcacccaatcacttgaaaaaggcaaaactcatgttcttttacacccgctaccccagctccaacatgctgaagacctacttctcggacgtgaagttcaacagatgcattacctcgcagctcatcaagtggttcagcaatttccgtgagttttactatatccagatggagaagtatgcgcgtcaagccatcaatgatggagtcaccagtacagaagagctctccatcaccagggattgtgagctataccgagccctcaacatgcactacaacaaagcaaatgactttgaggttccagagagattcctggaagttgcgcagatcacgttacgggagtttttcaatgccatcatcgcgggcaaagatgttgatccttcctggaagaaggccatttacaaggtcatctgcaagctggatagtgaagttcctgagattttcaaatcccctaactgcctacaagaactccttcacgagtag

SEQ ID NO: 3 - protein, 365 aa, synthetic construct
MELDFGHFDERDKASRNMRGSRMNGLPSPTHSAHCSFYRTRTLQALSNEKKAKKVRFYRNGDRYFKGIVYAVSSDRFRSFDALLADLTRSLSDNINLPQGVRYIYTIDGSRKIGSMDELEEGESYVCSSDNFFKKVEYTKNVNPNWSVNVKTSANMKAPQSLASSNSAQARENKDFVRPKLVTIIRSGVKPRKAVRVLLNKKTAHSFEQVLTDITEAIKLETGVVKKLYTLDGKQVTCLHDFFGDDDVFIACGPEKFRYAQDDFSLDENECRVMKGNPSAAAGPKASPTPQKTSAKSPGPMRRSKSPADSGNDQDANGTSSSQLSTPKSKQSPISTPTSPGSLRKHKDLYLPLSLDDSDSLGDSM

SEQ ID NO: 4 - DNA, 1101 bp, synthetic construct
  source 1..1101: mol_type = other DNA
atggaacttgattttggacattttgacgaacgagacaaagcatctagaaatatgagagggtcacggatgaatggacttccaagtcccactcatagtgcccactgtagcttctacagaaccagaaccttgcaggcattaagtaatgagaagaaggccaagaaggtacgtttctaccgcaatggggaccgttacttcaaggggattgtgtacgctgtttcttctgaccgttttcgtagttttgatgcgttgctggctgacctgacccgatccttgtctgacaacattaacctgcctcagggagtgcgctacatttataccattgacggatccaggaagattggaagcatggatgaactggaagaaggggaaagctatgtctgctcctcagacaacttctttaaaaaggttgagtacaccaagaatgtcaaccccaactggtctgtcaacgtaaagacatctgccaacatgaaagccccccagtccttggctagcagcaacagtgctcaagccagagagaacaaggactttgtgcgccccaaacttgtgaccatcattcgcagcggggtgaagccacggaaggctgtgcgcgtgcttctcaacaagaaaacagcccactctttcgagcaggtcctgactgacatcacagaagcgatcaaactggaaaccggagttgtcaaaaaactctacacccttgatggaaagcaggtcacctgtctccatgatttctttggtgatgatgatgtgttcattgcttgtggtcctgaaaaattccgctatgctcaagatgatttctccttggatgagaatgaatgcagagtcatgaaagggaatccatctgccgcagctggcccaaaggcttccccaacacctcaaaagacatctgctaaaagcccaggcccaatgcgccgcagcaagtctccagctgactcaggtaacgaccaagacgcaaatggaacttccagcagtcagctctcaacacctaagtcaaagcagtctcctatctctacacccacaagccctggaagtctgcggaagcacaaggtagacctgtacctgccgctgtcattggatgactctgattcacttggcgattccatgtga

SEQ ID NO: 5 - DNA, 22 bp, synthetic construct
  source 1..22: mol_type = other DNA
tccactctactccctggtattc

SEQ ID NO: 6 - DNA, 20 bp, synthetic construct
  source 1..20: mol_type = other DNA
tggccaagttgagcagatag

SEQ ID NO: 7 - DNA, 22 bp, synthetic construct
  source 1..22: mol_type = other DNA
agggagtgcgctacatttatac

SEQ ID NO: 8 - DNA, 22 bp, synthetic construct
  source 1..22: mol_type = other DNA
gttgtctgaggagcagacatag

SEQ ID NO: 9 - DNA, 20 bp, synthetic construct
  source 1..20: mol_type = other DNA
gtgatggcagcctcttatgt

SEQ ID NO: 10 - DNA, 19 bp, synthetic construct
  source 1..19: mol_type = other DNA
gggcttgtcccttgagttt

SEQ ID NO: 11 - DNA, 20 bp, synthetic construct
  source 1..20: mol_type = other DNA
gtgtgtgacgttcccattag

SEQ ID NO: 12 - DNA, 19 bp, synthetic construct
  source 1..19: mol_type = other DNA
tgtccattgaggtggagag

SEQ ID NO: 13 - DNA, 20 bp, synthetic construct
  source 1..20: mol_type = other DNA
cactcacctgctgctactca

SEQ ID NO: 14 - DNA, 20 bp, synthetic construct
  source 1..20: mol_type = other DNA
cttcttggggtcagcacaga

SEQ ID NO: 15 - DNA, 22 bp, synthetic construct
  source 1..22: mol_type = other DNA
caccagggattgtgagctatac

SEQ ID NO: 16 - DNA, 18 bp, synthetic construct
  source 1..18: mol_type = other DNA
acgtgatctgcgcaactt

SEQ ID NO: 17 - DNA, 19 bp, synthetic construct
  source 1..19: mol_type = other DNA
gcactctactgcgcatctt

SEQ ID NO: 18 - DNA, 22 bp, synthetic construct
  source 1..22: mol_type = other DNA
caccagactgtacgatcctttc